(19) United States
(12) Patent Application Publication
(10) Pub. No.: US 2024/0076263 A1
(43) Pub. Date: Mar. 7, 2024

(54) METHOD FOR PREPARING CHIRAL NITRO DERIVATIVES USING ORGANIC CHIRAL CATALYST COMPOUNDS AND ECO-FRIENDLY SOLVENTS

(71) Applicant: KOREA UNIVERSITY RESEARCH AND BUSINESS FOUNDATION, Seoul (KR)

(72) Inventors: Hyeon Soo KIM, Seoul (KR); Jae Ho SHIM, Seoul (KR)

(21) Appl. No.: 18/220,079

(22) Filed: Jul. 10, 2023

(30) Foreign Application Priority Data
Aug. 11, 2022 (KR) 10-2022-0100878

(51) Int. Cl. C07C 201/12; B01J 31/02; C07C 205/45; C07D 209/12; C07D 307/46; C07D 309/30; C07D 333/22; C07D 335/02

(52) U.S. Cl. CPC C07C 201/12; B01J 31/0271; C07C 205/45; C07D 209/12; C07D 307/46; C07D 309/30; C07D 333/22; C07D 335/02; B01J 2231/32; B01J 2531/007; C07B 2200/07; C07C 2601/08; C07C 2601/14

(57) ABSTRACT

Provided are a method for preparing chiral nitro derivatives using organic chiral catalyst compounds and water as an eco-friendly solvent, and the like. The catalyst is an organic catalyst based on (R,R)-1,2-diphenylethylenediamine (DPEN), and can prepare nitro derivatives having enantioselectivity and diastereoselectivity in excellent yield through a hydrophobic hydration effect. In particular, the preparation method of the present disclosure can stabilize a transition state through an interfacial reaction between the catalyst and water. In addition, indole derivatives can be synthesized using the chiral nitro derivatives prepared according to the present disclosure to be usefully used for the prevention or treatment of brain-nervous system diseases including depression and muscular diseases including cachexia.

### CROSS-REFERENCE TO RELATED APPLICATION

This application claims the benefit of Korean Patent Application No. 10-2022-0100878 filed on Aug. 11, 2022, in the Korean Intellectual Property Office, the entire disclosure of which is incorporated herein by reference for all purposes.

### BACKGROUND

### 1. Field of the Invention

The present disclosure relates to a method for preparing chiral nitro derivatives using organic chiral catalyst compounds and eco-friendly solvents, a method for preparing non-natural indole derivatives using the same, and the like.

### 2. Description of the Related Art

In understanding the basic properties of molecules, the spatial arrangement of atoms in a molecule has an important meaning. Over the last few decades, organic chemists have made many efforts to develop stereoselective organic reactions, and until recently, metal catalysts were used in significant amounts for asymmetric synthesis.

Metal catalysts exhibit high catalytic activity, but are expensive, and often unstable in reaction environments because metal ions contain air and moisture. In addition, when a metal catalyst is used, a small amount of metal may remain in products, which may cause environmental pollution. Therefore, in order to overcome these disadvantages, research on stereoselective synthesis using an organic catalyst has attracted attention.

Heterocyclic compounds are one of a large and diverse group of molecular fragments used by chemists for organic synthesis. The heterocyclic compounds are identified as special structures in medicinal chemistry and are known to have various pharmacological activities. Heterocyclic compounds with high enantioselectivity have been mainly synthesized through general organic synthesis reactions. However, conventional organic synthesis reactions require a large amount of metal or oxidizing agent, which is not economical and causes environmental problems by emitting by-products. In addition, since a reaction substrate is limited and other sensitive functional groups are not preserved during a reaction, it is difficult to synthesize (R)-type derivatives by conventional methods.

A Michael addition reaction is one of the most important carbon-carbon bond formation methods and is an important reaction used to synthesize a wide range of natural products or complex compounds that exhibit biological activity.

Under this background, the present inventors developed a method for producing nitro derivatives in an eco-friendly manner with high yield and optical purity by using (R,R)-1,2-diphenylethylenediamine (DPEN) as the basic skeleton of a chiral catalyst to introduce thiourea and applying α,β-unsaturated nitro compounds and aldehyde or ketone to a Michael addition reaction using hydrogen bond catalysis, and then completed the present disclosure.

### SUMMARY

An object of the present disclosure is to provide a method for preparing chiral nitro derivatives through an asymmetric Michael addition reaction using a chiral thiourea catalyst.

Another object of the present disclosure is to provide chiral nitro derivatives prepared by the preparation method.

Yet another object of the present disclosure is to provide a method for preparing chiral indole derivatives using the chiral nitro derivatives.

Still another object of the present disclosure is to provide chiral indole derivatives prepared by the preparation method.

However, technical objects of the present disclosure are not limited to the aforementioned purpose and other objects which are not mentioned may be clearly understood by those skilled in the art from the following description.

According to an aspect, there is provided a method for preparing chiral nitro derivatives including preparing a compound represented by Chemical Formula 3 by reacting a compound represented by Chemical Formula 1 with a compound represented by Chemical Formula 2 in water, in which in the reaction, a catalyst compound represented by Chemical Formula 4 is used:

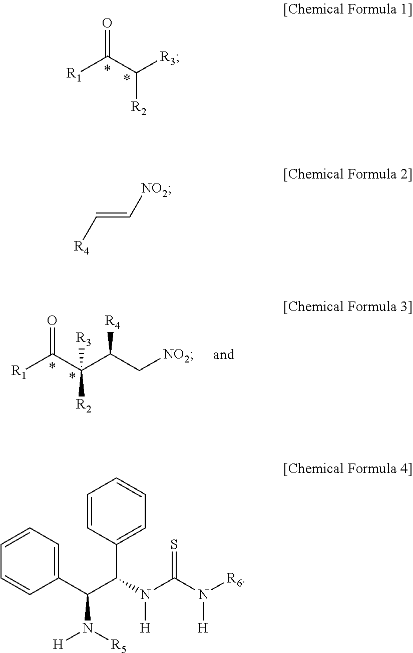

In Chemical Formulas 1 and 3, R₁ is hydrogen, a C₁-C₇ acyclic alkyl group, or a C₄-C₁₀ aryl group, and R₂ is hydrogen or a C₁-C₇ acyclic alkyl group, or the R₁ and R₂ may form a C₄-C₁₀ cycloalkyl group or heterocycloalkyl group together with the carbons to which the R₁ and R₂ are attached and carbons marked with asterisks (*), and R₃ is hydrogen or a C₁-C₇ acyclic alkyl group (however, except when R₁, R₂, and R₃ are all hydrogens),

in Chemical Formulas 2 and 3, R₄ is a C₄-C₁₀ aryl group or C₄-C₁₀ heteroaryl group unsubstituted or substituted with at least one selected from the group consisting of a halogen group, a hydroxyl group, a nitro group, a C₁-C₇ acyclic alkyl group, a C₁-C₇ alkoxy group, and combinations thereof, and

in Chemical Formula 4, R₅ is hydrogen or a C₁-C₇ acyclic alkyl group, and R₆ may be a C₄-C₁₀ aryl group or C₁-C₇ acyclic alkyl group unsubstituted or substituted with at least one selected from the group consisting of a halogen group, a cyano group, a nitro group, a trifluoromethyl group, a C₁-C₇ acyclic alkyl group, a C₁-C₇ alkoxy group, and combinations thereof.

As an embodiment of the present disclosure, the reaction may be an asymmetric Michael addition reaction.

As another embodiment of the present disclosure, the compound represented by Chemical Formula 1 may be at least one selected from the group consisting of compounds represented by Chemical Formulas 1-1 to 1-11:

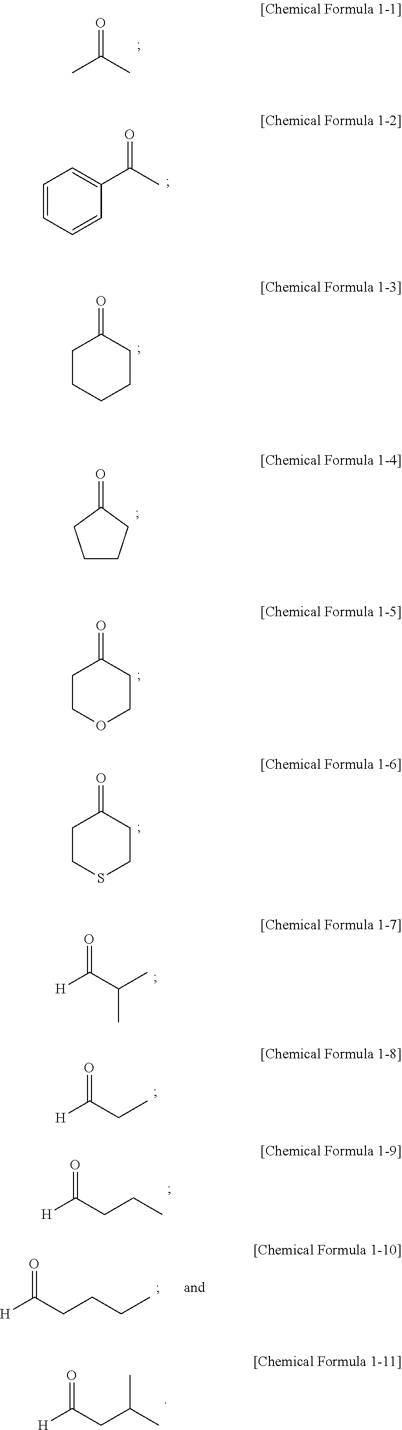

As another embodiment of the present disclosure, the compound represented by Chemical Formula 2 may be at least one selected from the group consisting of compounds represented by Chemical Formulas 2-1 to 2-14:

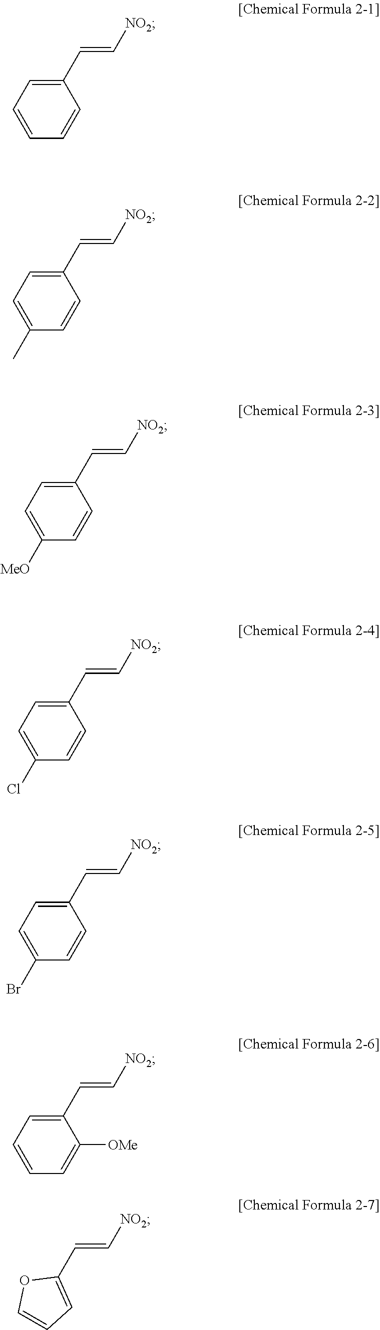

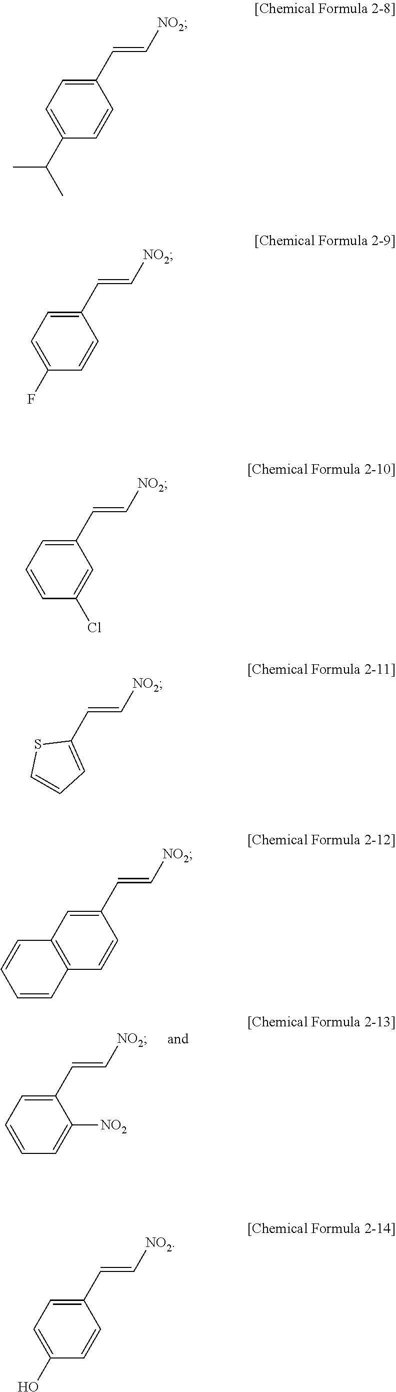

As another embodiment of the present disclosure, the compound represented by Chemical Formula 3 may be at least one selected from the group consisting of compounds represented by Chemical Formulas 3-1 to 3-36:

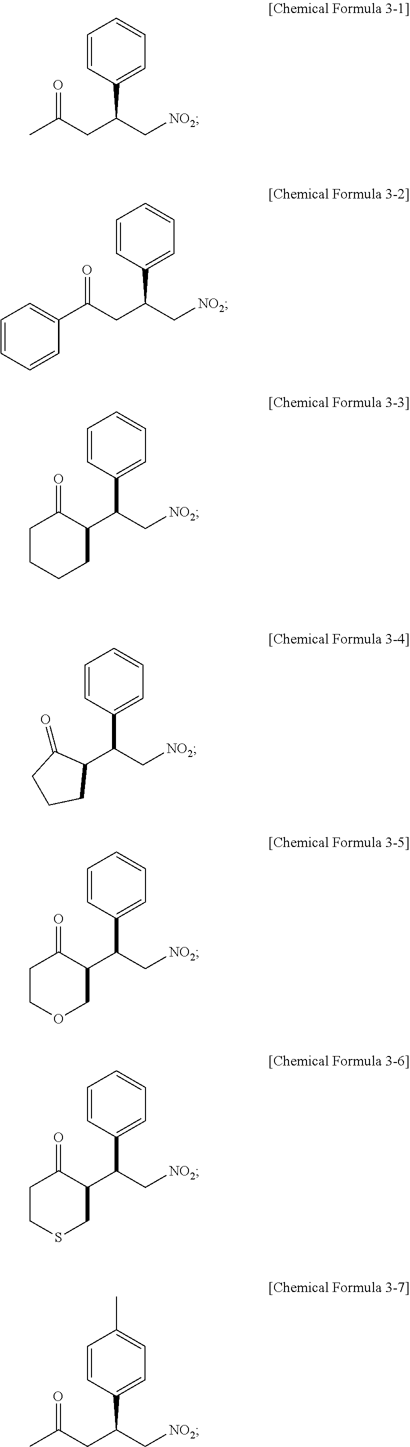

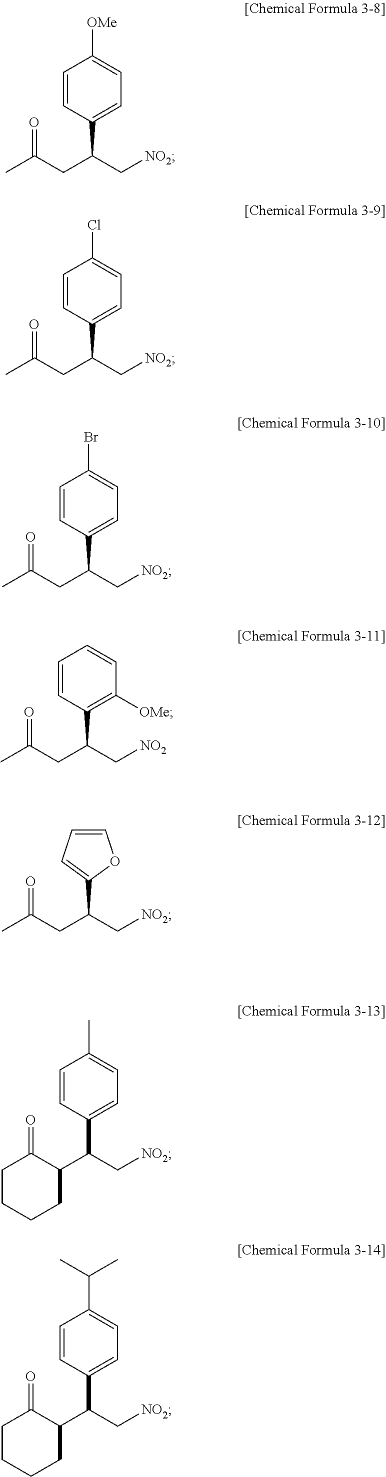

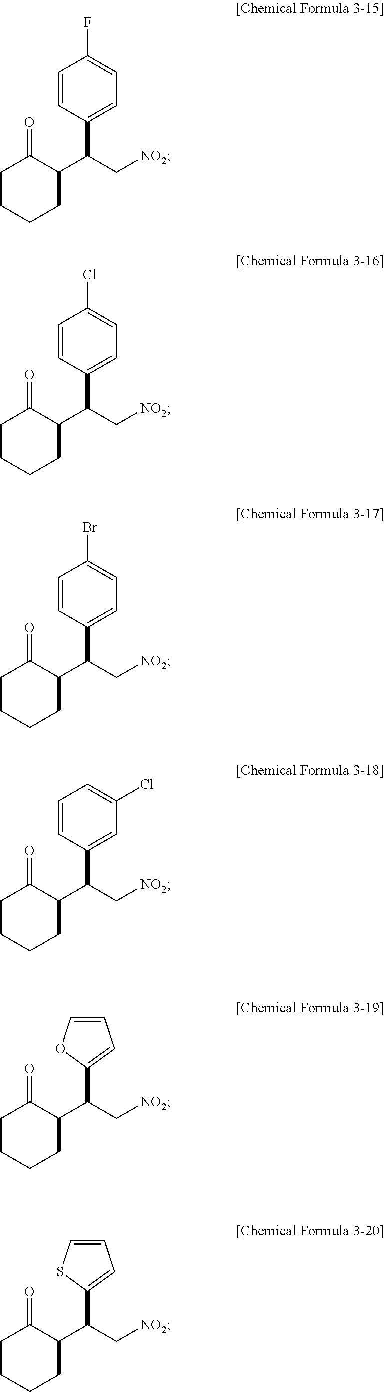

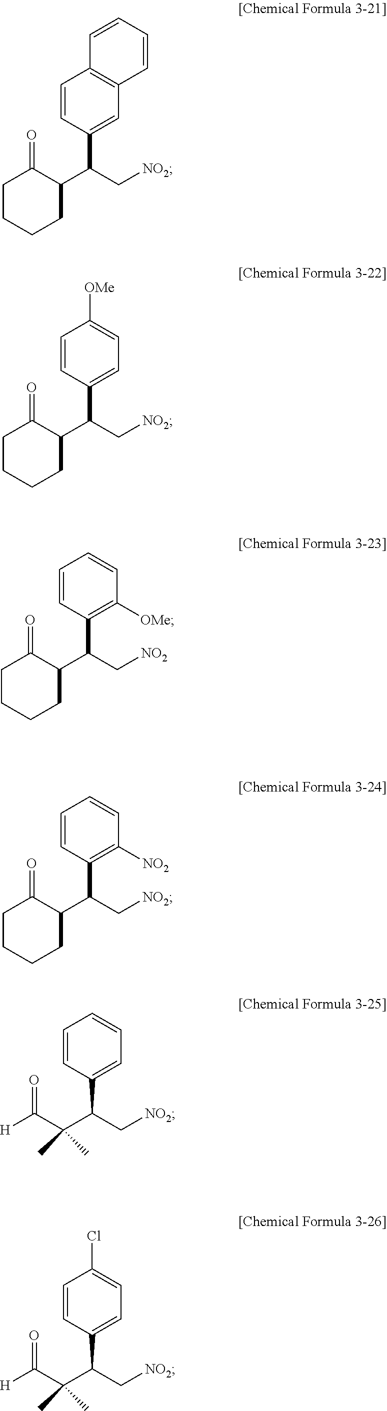

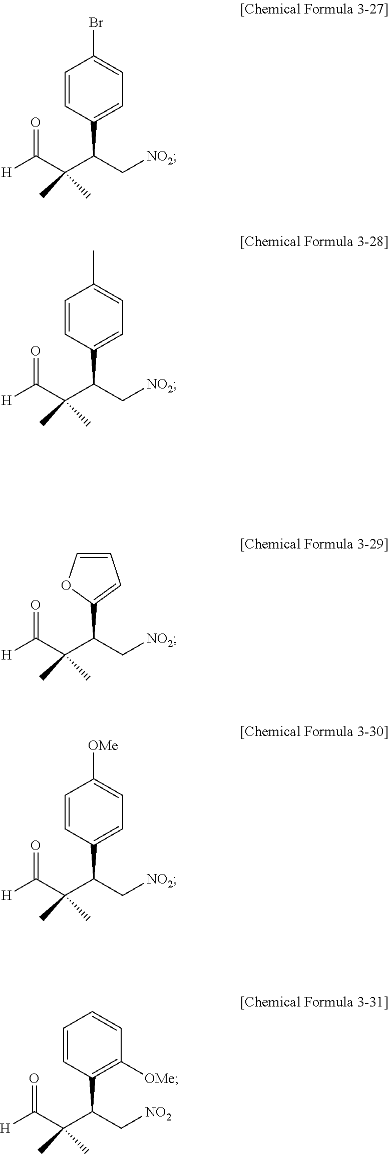

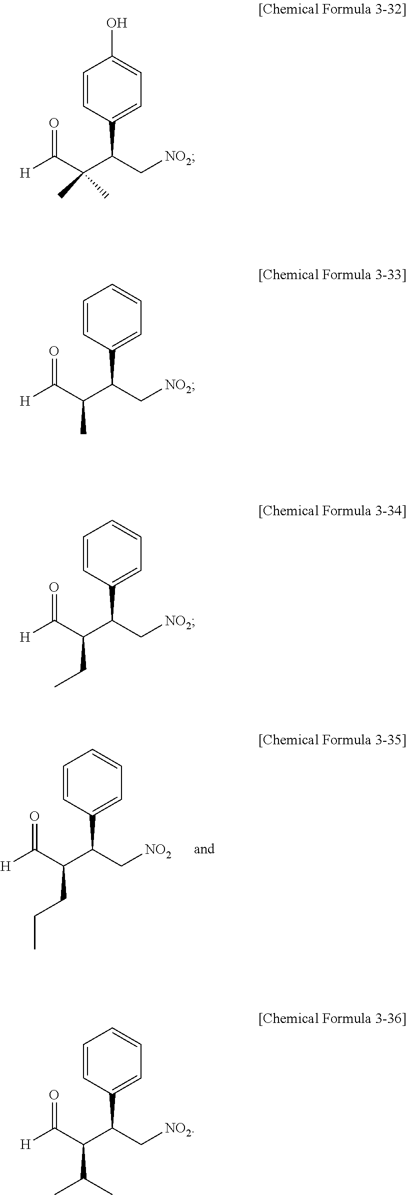

As another embodiment of the present disclosure, the compound represented by Chemical Formula 4 may be at least one selected from the group consisting of compounds represented by Chemical Formulas 4-1 to 4-9:

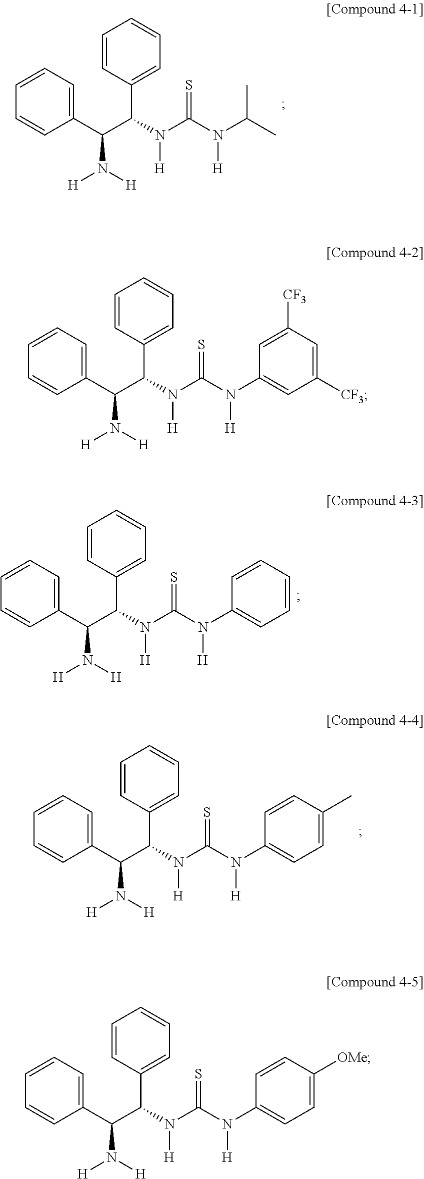 [SMILES: CC(C)NC(=S)N[C@@H](c1ccccc1)[C@@H](N)c1ccccc1 ; N[C@@H](c1ccccc1)[C@@H](NC(=S)Nc1cc(C(F)(F)F)cc(C(F)(F)F)c1)c1ccccc1 ; N[C@@H](c1ccccc1)[C@@H](NC(=S)Nc1ccccc1)c1ccccc1 ; Cc1ccc(NC(=S)N[C@@H](c2ccccc2)[C@@H](N)c2ccccc2)cc1 ; COc1ccc(NC(=S)N[C@@H](c2ccccc2)[C@@H](N)c2ccccc2)cc1]

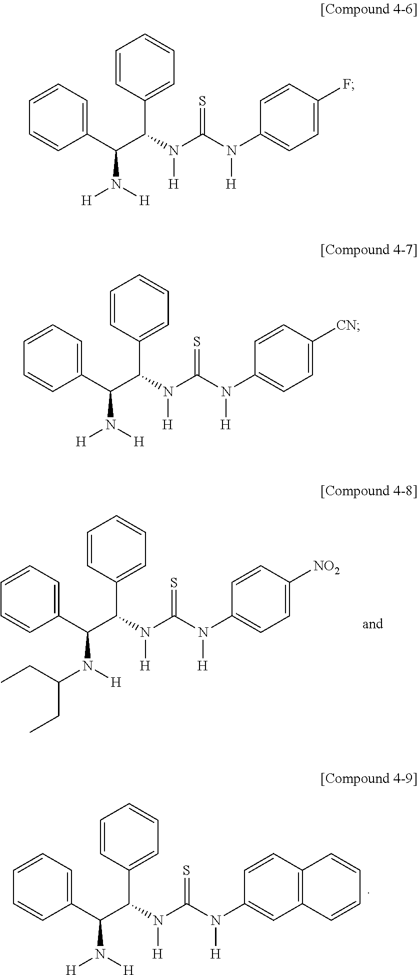 [SMILES: N[C@@H](c1ccccc1)[C@@H](NC(=S)Nc1ccc(F)cc1)c1ccccc1 ; N#Cc1ccc(NC(=S)N[C@@H](c2ccccc2)[C@@H](N)c2ccccc2)cc1 ; CCC(CC)N[C@@H](c1ccccc1)[C@@H](NC(=S)Nc1ccc([N+](=O)[O-])cc1)c1ccccc1 ; N[C@@H](c1ccccc1)[C@@H](NC(=S)Nc1ccc2ccccc2c1)c1ccccc1]

As another embodiment of the present disclosure, the compound represented by Chemical Formula 1 and the compound represented by Chemical Formula 2 may react with each other by further adding 5 mol % of 4-nitrophenol, phenol, or 4-chlorophenol, preferably 4-nitrophenol.

As another embodiment of the present disclosure, the reaction may be performed in one reactor at room temperature.

As another embodiment of the present disclosure, 5 to 15 equivalents, preferably 10 equivalents of the compound represented by Chemical Formula 1 and 0.5 to 2 equivalents, preferably 1 equivalent of the compound represented by Chemical Formula 2 may react with each other. At this time, 1 to 10 mol % of the catalyst compound represented by Chemical Formula 4 may be added.

In addition, the present disclosure provides chiral nitro derivatives prepared by the preparation method described above.

In addition, the present disclosure provides a method for preparing chiral indole derivatives including preparing a compound represented by Chemical Formula 5 below from the chiral nitro derivatives.

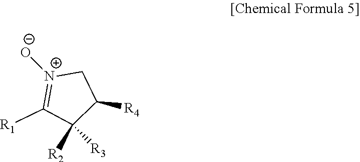

In Chemical Formula 5, R₁ is hydrogen, a C₁-C₇ acyclic alkyl group, or a C₄-C₁₀ aryl group, and R₂ is hydrogen or a C₁-C₇ acyclic alkyl group, or the R₁ and R₂ may form a C₄-C₁₀ cycloalkyl group or heterocycloalkyl group together with the carbons to which the R₁ and R₂ are attached and carbons marked with asterisks (*), and R₃ is hydrogen or a C₁-C₇ acyclic alkyl group (however, except when R₁, R₂, and R₃ are all hydrogens). R₄ is a C₄-C₁₀ aryl group or C₄-C₁₀ heteroaryl group unsubstituted or substituted with at least one selected from the group consisting of a halogen group, a hydroxyl group, a nitro group, a C₁-C₇ acyclic alkyl group, a C₁-C₇ alkoxy group, and combinations thereof.

As an embodiment of the present disclosure, in the chiral nitro derivatives, when R₁ and R₂ form a cyclohexyl group together with the carbons to which the R₁ and R₂ are attached and carbons marked with asterisks (*), the indole derivative may be a compound represented by Chemical Formula 5′.

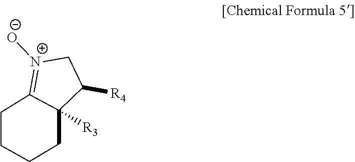

As another embodiment of the present disclosure, the chiral indole derivatives may be prepared by adding Zn powder and NH₄Cl to the chiral nitro derivatives. At this time, the solvent may be THE or water, and reacted at room temperature for 6 hours.

In addition, the present disclosure provides chiral indole derivatives prepared by the preparation method described above.

Additional aspects of embodiments will be set forth in part in the description which follows and, in part, will be apparent from the description, or may be learned by practice of the disclosure.

According to the embodiments, by the method for preparing the chiral nitro derivatives, nitro derivatives having enantioselectivity and diastereoselectivity can be prepared through hydrophobic hydration using a hydrophobic organic catalyst that can be easily synthesized from (R,R)-1,2-diphenylethylenediamine (DPEN). In addition, the catalyst may maintain yield and optical purity at predetermined levels or higher even when reused 4 or more times.

According to the embodiments, the method for preparing the chiral nitro derivatives is eco-friendly by using water as a solvent, and stabilizes a transition state through the interfacial reaction between the solvent and the catalyst compound, thereby improving the yield and optical purity of the chiral nitro derivatives as products and reducing the reaction time.

According to the embodiments, since the chiral nitro derivatives are used as intermediates in the synthesis of indole and the like, which are used as physiologically active substances, indole derivatives prepared using the chiral nitro derivatives can be usefully used for the prevention or treatment of brain-nervous system diseases including depression and muscular diseases including cachexia.

The brain-nervous system diseases may include depression, hyperactivity, attention deficit, autism, anxiety disorder, sleep disorder, panic disorder, intellectual disorder, memory loss, drug addiction, schizophrenia, obsessive-compulsive disorder, delusion of grandeur, personality disorder, alcoholism, bipolar disorder, etc., but are not limited thereto.

The muscular diseases may include cachexia, sarcopenia, atony, muscular atrophy, muscular dystrophy, myasthenia, muscle degeneration, muscle stiffness, amyotrophic lateral sclerosis, myositis, muscle calcification, muscle ossification, etc., but are not limited thereto.

Effects of the chiral nitro derivatives and the preparation method thereof according to the embodiment of the present disclosure are not limited to those mentioned above, and other effects not mentioned will be clearly understood by those skilled in the art from the following description.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates Reaction Formula of a method for preparing chiral nitro derivatives and indole derivatives according to an embodiment of the present disclosure. Ketone or aldehyde (Chemical Formula 1) and nitroalkene (Chemical Formula 2) react with a thiourea catalyst (Chemical Formula 4) in water (H₂O) to prepare a chiral nitro derivative (Chemical Formula 3) and prepare an indole derivative (Chemical Formula 5) using the same;

FIG. 2 illustrates possible stereochemical transition state models and expected syn and anti products for a Michael reaction of cyclohexanone and phenyl-substituted nitroalkene;

FIG. 3 illustrates a catalytic mechanism proposed based on B3LYP/6-31G(d,p) calculations and a free energy diagram of an enantioselective Michael reaction of (R,R)-1,2-diphenylethylenediamine (DPEN)-thiourea-catalyzed cyclohexanone and phenyl-substituted nitroalkene;

FIG. 4 illustrates possible stereochemical transition state models and expected syn and anti products for a Michael reaction of propionaldehyde and phenyl-substituted nitroalkene;

FIG. 5 illustrates a catalytic mechanism proposed based on B3LYP/6-31G(d,p) calculations and a free energy diagram of an enantioselective Michael reaction of (R,R)-1,2-diphenylethylenediamine (DPEN)-thiourea-catalyzed propionaldehyde and phenyl-substituted nitroalkene;

FIG. 6 illustrates a catalytic mechanism proposed based on B3LYP/6-31G(d,p) calculations and a relative free energy diagram of an (R,R)-1,2-diphenylethylenediamine (DPEN)-thiourea-catalyzed enantioselective Michael reaction, in which the calculations were performed using water, toluene, and an aqueous binary mixture (solvent+H₂O) conditions;

FIG. 7 illustrates a catalytic mechanism proposed based on B3LYP/6-31G(d,p) calculations and a relative free energy diagram of an (R,R)-1,2-diphenylethylenediamine (DPEN)-thiourea-catalyzed enantioselective Michael reaction, in which the calculations were performed using water, ethanol (EtOH), dichloromethane (CH₂Cl₂), tetrahydrofuran (THF), chloroform, toluene, benzene, and n-hexene as solvents;

FIG. 8 illustrates a catalytic mechanism proposed based on B3LYP/6-31G(d,p) calculations and a free energy diagram of an enantioselective Michael reaction of (R,R)-1,2-diphenylethylenediamine (DPEN)-thiourea-catalyzed isobutyraldehyde and phenyl-substituted nitroalkene; and

FIG. 9A and FIG. 9B illustrate results of nuclear magnetic resonance spectroscopy (NMR) to confirm hydrogen bonds between fluorine (F) atoms and hydrogen (H) atoms in a Michael addition reaction using an organic chiral catalyst 4-2 and water as a solvent. FIG. 9A illustrates an NMR result when there is water (D₂O) and FIG. 9B illustrates an NMR result when there is no water.

### DETAILED DESCRIPTION

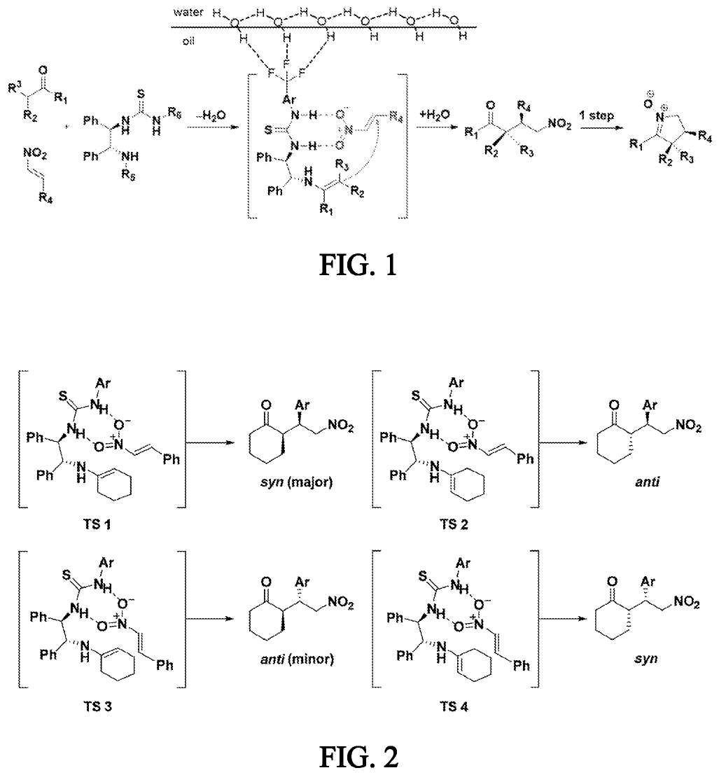

The present inventors applied a thiourea catalyst based on (R,R)-1,2-diphenylethylenediamine (DPEN) to an asymmetric Michael addition reaction of nitroalkene and aldehyde or ketone in water to prepare chiral nitro derivatives having a high level of enantioselectivity (76 to 99% syn ee) and diastereoselectivity (syn/anti=9/1) in an eco-friendly manner in a high yield of 88% to 99% (FIG. 1).

Specifically, a primary amine moiety of DPEN reacts with carbonyl to form enamine and is activated through the formation of hydrogen bonds between nitro groups of α,βunsaturated nitroalkene and thiourea. That is, an asymmetric Michael product was obtained by 1,4-adding enamine to alkene to form a new carbon-carbon bond. At this time, nitro derivatives as a product may have high stereoselectivity through double activation by hydrogen bonds between nitro groups and thiourea.

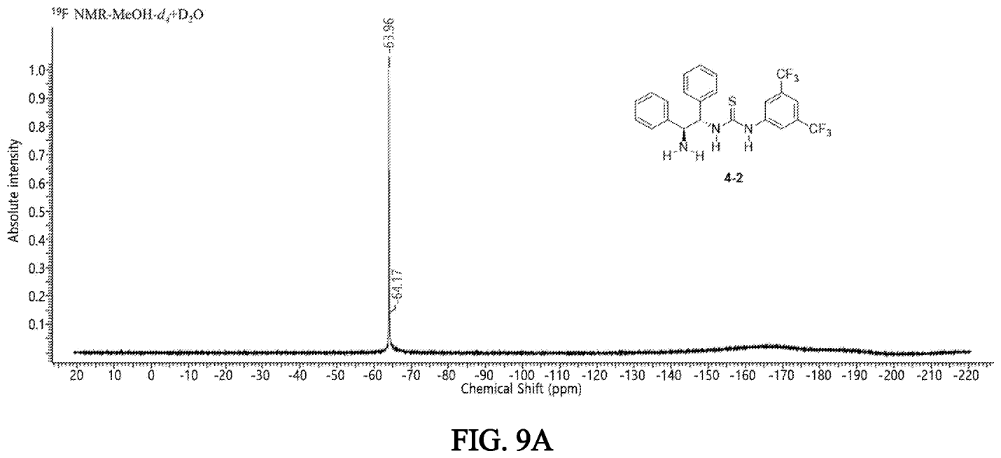

As an embodiment of the present disclosure, in the case of the Michael addition reaction using isobutyraldehyde and α,β-unsaturated nitroalkene, the hydrogen of a thiourea moiety of the catalyst forms a hydrogen bond with the oxygen atom of the nitroalkene, and an amine moiety of the catalyst reacts with aldehyde to form enamine. Subsequently, the enamine, which is a nucleophile, approaches the rear surface of the α,β-unsaturated nitroalkene to produce a compound with predominant stereoselectivity for an (R)-product (FIG. 8).

Accordingly, the present disclosure provides a method for preparing chiral nitro derivatives including preparing a compound represented by Chemical Formula 3 by reacting a compound represented by Chemical Formula 1 and a compound represented by Chemical Formula 2 in water using a catalyst compound represented by Chemical Formula 4:

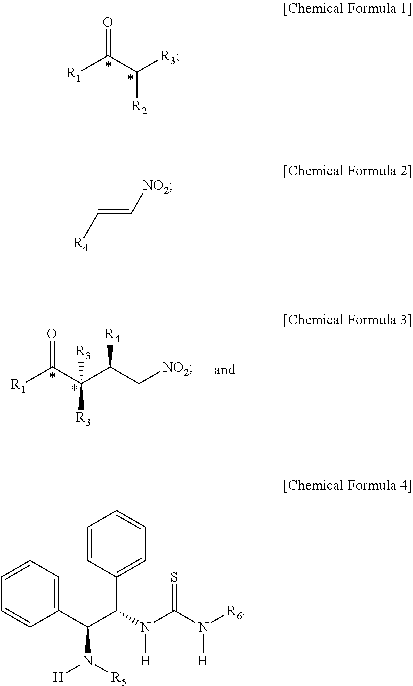

In Chemical Formulas 1 and 3, R₁ is hydrogen, a C₁-C₇ acyclic alkyl group, or a C₄-C₁₀ aryl group, and R₂ is hydrogen or a C₁-C₇ acyclic alkyl group, or the R₁ and R₂ may form a C₄-C₁₀ cycloalkyl group or heterocycloalkyl group together with the carbons to which the R₁ and R₂ are attached and carbons marked with asterisks (*), and R₃ is hydrogen or a C₁-C₇ acyclic alkyl group (however, except when R₁, R₂, and R₃ are all hydrogens), in Chemical Formulas 2 and 3, R₄ is a C₄-C₁₀ aryl group or C₄-C₁₀ heteroaryl group unsubstituted or substituted with at least one selected from the group consisting of a halogen group, a hydroxyl group, a nitro group, a C₁-C₇ acyclic alkyl group, a C₁-C₇ alkoxy group, and combinations thereof, and in Chemical Formula 4, R₅ is hydrogen or a C₁-C₇ acyclic alkyl group, and R₆ may be a C₄-C₁₀ aryl group or C₁-C₇ acyclic alkyl group unsubstituted or substituted with at least one selected from the group consisting of a halogen group, a cyano group, a nitro group, a trifluoromethyl group, a C₁-C₇ acyclic alkyl group, a C₁-C₇ alkoxy group, and combinations thereof.

In the present disclosure, the term “substitution” is a reaction in which atoms or atom groups included in molecules of a compound are replaced with other atoms or atom groups.

In the present disclosure, the term “acyclic alkyl group” refers to a group derived from straight-chain or branched-chain saturated aliphatic hydrocarbon having a specified number of carbon atoms and having at least one valency. Examples of these alkyl groups include methyl, ethyl, propyl, isopropyl, butyl, isobutyl, 2-butyl, 3-butyl, pentyl, n-hexyl, and the like, but are not limited thereto.

In the present disclosure, the term “cycloalkyl group” is also referred to as a cyclic alkyl group, and refers to a monovalent group having at least one saturated ring in which all ring members are carbons. Examples of such a cycloalkyl group include a cyclobutyl group, a cyclopentyl group, a cyclohexyl group, and the like, but are not limited thereto.

In the present disclosure, the term “heterocycloalkyl group” usually refers to saturated or unsaturated (but not aromatic) cyclohydrocarbon, which may optionally be unsubstituted, monosubstituted, or polysubstituted, and in its structure, at least one is selected from the group consisting of heteroatoms of N, O, or S.

In the present disclosure, the term “aryl group” refers to an unsaturated aromatic ring compound having 6 to 20 carbon atoms having a single ring (e.g., phenyl) or a plurality of condensed rings (e.g., naphthyl). Examples of such aryl include phenyl, naphthyl, and the like, but are not limited thereto.

In the present disclosure, the term “heteroaryl group” refers to a single ring or a plurality of condensed rings in which at least one among atoms constituting the ring has a heteroatom of N, O, or S. Examples of such a heteroaryl group include a furyl group, a thiophenyl group, a pyridyl group, a pyrimidinyl group, a pyrazinyl group, an oxazolyl group, and the like, but are not limited thereto.

In the present disclosure, the term “alkoxy group” refers to an atom group CₙH₂ₙ₊₁O⁻ formed by bonding an oxygen atom to an alkyl group, and examples of such an alkoxy group include methoxy, ethoxy, propoxy, phthaloxy, and the like, but are not limited thereto.

In the present disclosure, the term “halogen group” may be fluorine (F), chloride (C₁), bromine (Br), iodine (I), or the like, as elements belonging to Group 17 of the periodic table.

In addition, since the chiral nitro derivatives of the present disclosure may be economically prepared without using expensive metal catalysts unlike conventional preparation methods and use water as an eco-friendly solvent under mild reaction conditions, the chiral indole derivatives prepared using the chiral nitro derivatives may be included in pharmaceutical compositions used for the treatment or prevention of various diseases.

Although the type of the disease is not limited, the chiral indole derivatives of the present disclosure may be used as an antidepressant and a cancer cachexia therapeutic agent, so that the present disclosure provides a composition for the prevention or treatment of brain-nervous system diseases including depression and muscular diseases including cachexia, including derivatives represented by Chemical Formula 5 below as an active ingredient.

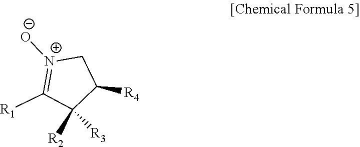

In Chemical Formula 5, R₁ is hydrogen, a C₁-C₇ acyclic alkyl group, or a C₄-C₁₀ aryl group, and R₂ is hydrogen or a C₁-C₇ acyclic alkyl group, or the R₁ and R₂ may form a C₄-C₁₀ cycloalkyl group or heterocycloalkyl group together with the carbons to which the R₁ and R₂ are attached and carbons marked with asterisks (*), and R₃ is hydrogen or a C₁-C₇ acyclic alkyl group (however, except when R₁, R₂, and R₃ are all hydrogens), and R₄ is a C₄-C₁₀ aryl group or C₄-C₁₀ heteroaryl group unsubstituted or substituted with at least one selected from the group consisting of a halogen group, a hydroxyl group, a nitro group, a C₁-C₇ acyclic alkyl group, a C₁-C₇ alkoxy group, and combinations thereof.

The pharmaceutical composition according to the present disclosure may include a pharmaceutically acceptable carrier in addition to the active ingredients. The pharmaceutically acceptable carrier is generally used in preparation, and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, mineral oil, and the like, but is not limited thereto. Further, the pharmaceutical composition may further include a lubricant, a wetting agent, a sweetening agent, a flavoring agent, an emulsifying agent, a suspending agent, a preservative, and the like, in addition to the ingredients.

The pharmaceutical composition of the present disclosure may be administered orally or parenterally (e.g., applied intravenously, subcutaneously, intraperitoneally, or topically) according to a desired method, and a dose thereof varies depending on the condition and body weight of a patient, the severity of a disease, a drug form, and route and time of administration, but may be appropriately selected by those skilled in the art.

The pharmaceutical composition of the present disclosure is administered in a pharmaceutically effective dose. The ‘pharmaceutically effective dose’ used herein refers to an amount enough to treat diseases at a reasonable benefit/risk ratio applicable to medical treatment. The effective dose level may be determined according to factors including the type and severity of a disease, the activity of a drug, the sensitivity to a drug, a time of administration, a route of administration, an excretion rate, duration of treatment, and simultaneously used drugs in patients, and other factors well-known in the medical field. The pharmaceutical composition according to the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side-effects by considering all the factors, which may be easily determined by those skilled in the art.

Specifically, the effective dose of the pharmaceutical composition of the present disclosure may vary depending on the age, sex, condition, and body weight of a patient, absorption rate, inactivity rate, and excretion rate of active ingredients in the body, a disease type, and combined drugs.

As another aspect of the present disclosure, the present disclosure provides a method for the prevention or treatment of brain-nervous system diseases including depression and muscular diseases including cachexia, including administering the pharmaceutical composition to a subject. The “subject” used herein refers to a subject in need of treatment or prevention for diseases, and more particularly, refers to mammals such as humans or non-human primates, mice, dogs, cats, horses, and cattle.

The terms used in embodiments are used for the purpose of description only, and should not be construed to be limited. The singular expression includes the plural expression unless the context clearly dictates otherwise. In the present specification, it should be understood that term “comprising” or “having” indicates that a feature, a number, a step, an operation, a component, a part, or the combination thereof described in the specification is present, but does not exclude a possibility of presence or addition of one or more other features, numbers, steps, operations, components, parts, or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present application.

In describing the components of the embodiments of the present disclosure, terms including first, second, A, B, (a), (b), and the like may be used. These terms are just intended to distinguish the components from other components, and the terms do not limit the nature, sequence, or order of the components. When it is disclosed that any component is “connected”, “coupled”, or “linked” to other components, it should be understood that the component may be directly connected or linked to other components, but another component may be “connected”, “coupled”, or “linked” between the respective components.

Hereinafter, embodiments will be described in detail with reference to the accompanying drawings. However, since various modifications may be made to the embodiments, the scope of the patent application is not limited or restricted by these embodiments. It should be understood that all modifications, equivalents, and substitutes for the embodiments are included in the scope of the present disclosure.

In addition, in the description with reference to the accompanying drawings, like components designate like reference numerals regardless of reference numerals and a duplicated description thereof will be omitted. In describing the embodiments, a detailed description of related known technologies will be omitted if it is determined that they unnecessarily make the gist of the embodiments unclear.

The present disclosure may have various modifications and various embodiments and specific embodiments will be illustrated in the drawings and described in detail in the detailed description. However, this does not limit the present disclosure within specific embodiments, and it should be understood that the present disclosure covers all the modifications, equivalents, and replacements within the idea and technical scope of the present disclosure. In the interest of clarity, not all details of the relevant art are described in detail in the present specification in so much as such details are not necessary to obtain a complete understanding of the present disclosure.

### EXAMPLE 1

### Synthesis of chiral nitro Derivatives of Present Disclosure

1.1. Instruments and Reagents

IR spectra were recorded using a NICOLET 380 FT-IR spectrophotometer (Thermo Fisher Scientific Inc., Waltham, MA, USA), and optical rotation was measured using an automatic digital polarimeter (model name: A20766 APV/6w, Rudolph Research Analytical, Hackettstown, NJ, USA). ¹H NMR and ¹³C NMR spectra were obtained based on the internal standard of TMS using Varian Gemini 300 (300, 75 MHz, Agilent, Santa Clara, CA, USA), Varian Mercury 400 (400, 100 MHz, Agilent, Santa Clara, CA, USA), and Bruker Avance 500 (500, 125 MHz, Bruker BioSpin GmbH, Silberstreifen 4, 76287 Rheinstetten, Billerica, MA, USA). Chiral HPLC analysis was performed using a Jasco LC-1500 Series HPLC system (JASCO, 4-21, Sennin-cho 2-chome, Hachioji, Tokyo 193-0835, Japan). All reactions were performed under an argon environment in a flask well-dried in an oven. Toluene (CaH₂), THF (Na, benzophenone), and CH₂Cl₂ (CaH₂) reaction solvents were purified before use. Reagents used herein were products from Aldrich (Louis, MO, USA), TCI (Tokyo, Japan), etc., and purified or dried by known methods, if necessary. Merck's silica gel 60 (230 to 400 mech) was used as a stationary phase for column chromatography.

### 1.2. Synthesis of thiourea Catalyst

(R,R)-1.2-diphenylethylenediamine (200 mg, 0.942 mmol) was dissolved in toluene (1.00 mL), and then added with isothiocyanate (0.140 mL, 0.942 mmol) and stirred at 0° C. for 1 hour. After completion of the reaction with distilled water, the mixture was extracted with dichloromethane (20 mL×3 times), dehydrated with MgSO₄, filtrated, and concentrated under reduced pressure, and then a product (Chemical Formula 4, however, R₅═H) was isolated using column chromatography (SiO₂, EtOAc:CH₂Cl₂=1:6) (Reaction Formula 1).

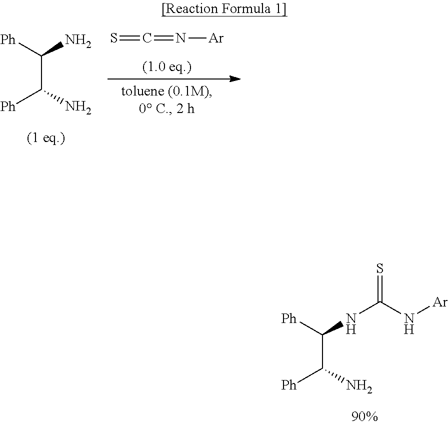

In addition, an N-monoalkylated thiourea catalyst was synthesized according to Reaction Formula 2 below.

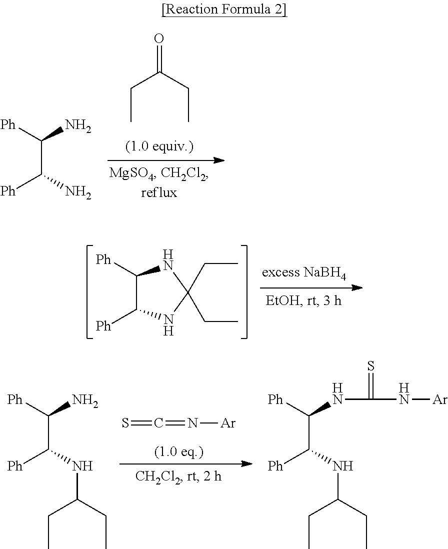

(R,R)-1,2-diphenylethylenediamine (1.0 equiv.) was dissolved in toluene (0.1 M), added with MgSO₄ and 3-pentanone (1.0 equiv.), and then heated and refluxed. After 48 hours, CH₂Cl₂ and MgSO₄ were filtered and removed, and the produced diaminoacetal was dissolved in ethanol, added with an excess of NaBH₄, and then stirred at room temperature for 3 hours. After the reaction was terminated with a 1 N NaOH aqueous solution, the mixture was extracted with CH₂Cl₂ three times. The extract was dehydrated with anhydrous MgSO₄, filtrated, and concentrated under reduced pressure, and then the residue thereof was subjected to column chromatography (SiO₂, CH₂Cl_{2:}MeOH:NH₃=300:10:1) to obtain a product. Mono-alkylated (R,R)-1,2-diphenylethylenediamine (1.0 equiv.) was dissolved in CH₂Cl₂ (0.2 M) under argon, added with isothiocyanate (1.1 equiv.), and then stirred at room temperature. After 2 hours, the mixture was transferred to water and extracted three times with 100 mL of CH₂Cl₂. The combined organic layers were dehydrated with anhydrous MgSO₄, filtered, and concentrated under reduced pressure, and the residue thereof was subjected to column chromatography (SiO₂, EA:hexane=1:5) to obtain a desired product (Chemical Formula 4, however, R₅=3-pentyl).

Non-limiting examples of the thiourea catalyst according to an embodiment of the present disclosure were as follows:

### (1) 1-[(1R,2R)-2-Amino-1,2-diphenylethyl]-3-isopropylthiourea (Chemical Formula 4-1)

[α]_{D}²⁵+62.3 (c 1.0, CHCl₃); ¹H-NMR (500 MHZ, CDCl₃) δ7.37-7.21 (m, 11H), 6.04 (br s, 1H), 5.09 (br s, 1H), 4.30 (s, 1H), 4.15 (br s, 1H), 1.73 (s, 2H), 1.09-1.03 (m, 6H) ppm; ¹³C-NMR (125 MHz, CDCl₃) δ180.68, 142.06, 129.00, 128.75, 127.88, 126.91, 64.12, 60.55, 46.39, 22.70 ppm; IR (KBr) 3335, 2966, 1527, 1326, 1273, 966, 695, 514 cm⁻¹; HRMS (ESI+) for C₁₈H2₃N₃S [M+H]⁺ Calcd: 314.1691, Found: 314.1627;

### (2) 1-[(1R,2R)-2-Amino-1,2-diphenylethyl]-3-[3,5-bis(trifluoromethyl)phenyl]thiourea (Chemical Formula 4-2)

[α]_{D}²⁵+13.5 (c 1.0, CH₃Cl); ¹H-NMR (500 MHz, DMSO-d6) δ8.25 (s, 2H), 7.78 (s, 1H), 7.32-7.15 (m, 13H), 5.99 (d, J=3 Hz, 1H), 4.77 (d, J=3 Hz, 1H) ppm; ¹³C-NMR (125 MHz, DMSO-d6) δ180.51, 143.26, 142.48, 130.82, 130.56, 128.51, 128.29 127.66, 127.55, 127.38, 124.78, 122.62, 121.34, 116.08, 63.66, 59.94 ppm; IR (KBr) 3305, 3032, 2963, 1652, 1601, 1557, 1383, 1277, 1262, 803, 700 cm⁻¹; HRMS (FAB⁺) for C₂₃H₁₉F₆N₃S [M+H]⁺ Calcd: 484.1282, Found: 484.1254;

### (3) 1-[(1R,2R)-2-Amino-1,2-diphenylethyl]-3-phenylthiourea (Chemical Formula 4-3)

[α]_{D}²⁰=+62.0 (c=0.02, CH₂Cl₂); ¹H-NMR (300 MHz, CDCl₃) δ7.76 (s, 1 H), 7.54-7.19 (m, 15H), 5.54 (s, 1H), 4.42 (d, 1H, J=5Hz), 1.35 (br s, 1H); ¹³C-NMR (100 MHz, DMSO-d6) δ182.09, 134.48, 133.93, 129.89, 128.70, 128.10, 127.91, 127.15, 126.94, 126.82, 126.74, 126.23, 125.59, 125.24, 122.98, 63.07, 59.09; IR (KBr) 3287.86, 3027.84, 1521.63, 1241.99, 1072.28, 939.20, 698.13 cm⁻¹; HRMS (FAB+) for C₂₁H₂₂N₃S [M+H]⁺ Calcd: 348.4918, Found: 348.1534.

### (4) 1-[(1R,2R)-2-Amino-1,2-diphenylethyl]-3-p-tolythiourea (Chemical Formula 4-4)

[α]_{D}²²+0.27 (c 1.00, CH₃Cl); ¹H-NMR (300 MHz, DMSO-d6) δ9.79 (s, 1H), 7.13-7.38 (m, 15H), 5.50(s, 1H), 4.32 (d, J=3 Hz, 1H), 2.27(s, 3H) ppm; ¹³C-NMR (100 MHz, DMSOd6) δ180.90, 143.72, 142.20, 137.14, 134.32, 129.86, 128.82 128.59, 127.68, 127.61, 127.46, 127.39, 123.97, 63.88, 60.00, 21.23 ppm; IR (KBr) 3301.69, 2861.98, 1889.99, 1527.42, 1342.28, 964.28, 701.99, 524.56 cm⁻¹; HRMS (FAB+) for C₂₂H₂₄N₃S [M+H]⁺ Calcd: 362.1691, Found: 362.2188

### (5) 1-[(1R,2R)-2-Amino-1,2-diphenylethyl]-3-(4-methoxyphenyl)thiourea (Chemical Formula 4-5)

[α]_{D}²²+0.327 (c 1.00, CH₃Cl); ¹H-NMR (300 MHz, DMSO-d6) δ9.55 (s, 1H), 8.17 (s, 1H), 7.12-7.22 (m, 12H), 6.87(d, J=6.0 Hz, 3H), 5.94 (s, 1H), 3.72 (s, 3H) ppm; ¹³C-NMR (100 MHz, DMSO-d6) δ180.72, 156.54, 139.70, 131.67, 128.09, 127.97, 127.19, 125.61, 113.93, 62.38, 59.81. 55.24 ppm; IR (KBr) 3303.63, 3027.84, 1733.78, 1510.06, 1297.92, 1243.92, 1029.85, 831.21, 700.07, 568.93 cm⁻¹; HRMS (FAB+) for C₂₂H₂₄N₃OS [M+H]⁺ Calcd: 378.1640, Found: 378.1563

### (6) 1-[(1R,2R)-2-Amino-1,2-diphenylethyl]-3-(4-fluorophenyl)thiourea (Chemical Formula 4-6)

[α]_{D}²²+0.132 (c 1.00, CH₃Cl); ¹H-NMR (300 MHz, DMSO-d6) δ9.93 (s, 1H), 7.08-7.46 (m, 15H), 5.52(s, 1H), 4.34 (d, J=3 Hz, 1H) ppm; ¹³C-NMR (100 MHz, DMSO-d6) δ181.28, 160.71, 143.73, 142.10, 136.32, 128.79, 128.58, 127.70, 127.59, 127.49, 127.39, 125.79, 115.94, 115.71, 63.88, 60.06 ppm; IR (KBr) 3301.69, 3029.77, 1874.56, 1527.42, 1342.27, 1218.85, 840.85, 701.99 cm⁻¹; HRMS (FAB+) for C₂₁H₂₁FN₃S [M+H]⁺ Calcd: 366.1440, Found: 366.1440

### (7) 1-[(1R,2R)-2-Amino-1,2-diphenylethyl]-3-(4-cyanophenyl)thiourea (Chemical Formula 4-7)

[α]_{D}²²+1.50 (c 1.00, CH₃Cl); ¹H-NMR (300 MHz, DMSO-d6) δ10.50 (s, 1H), 7.07-7.88 (m, 15H), 5.56 (d, J=3 Hz, 1H), 4.39 (d, J=3 Hz, 1H) ppm; ¹³C-NMR (100 MHz, DMSO-d6) δ180.47, 145.05, 143.34, 141.61, 133.36, 128.83, 128.64 127.74, 127.68, 127.59, 127.50, 121.37, 119.85, 105.11, 63.74, 60.04 ppm; IR (KBr) 3263.1, 2219.7, 1646.9, 1592.9, 1361.5, 1091.5 cm⁻¹; HRMS (FAB+) for C₂₂H₂₀N₄S [M+H]⁺ Calcd: 372.1487, Found: 372.1400

### (8) 1-(4-Nitrophenyl)-3-[(1R,2R)-2-(pentan-3-ylamino)-1,2-diphenylethyl]thiourea (Chemical Formula 4-8)

[α]_{D}²⁰+37.7 (c 0.20, CHCl₃); ¹H-NMR (300 MHz, DMSO-d6) δ10.5 (s, 1H), 8.16 (m, 2H), 7.90 (d, J=9.1 Hz, 2H), 7.37-7.15 (m, 10H), 5.54 (br s, 1H), 4.16 (d, J=5.5 Hz, 1H), 2.07 (m, 1H), 1.30-1.15 (m, 4H), 0.75 (t, J=7.4 Hz, 3H), 0.50 (t, J=7.4 Hz, 3H) ppm; ¹³C-NMR (100 MHz, DMSO-d6) δ179.85, 146.27, 141.82, 141.25, 140.25, 128.00, 127.86, 127.04, 126.98, 126.87, 124.60, 124.46, 120.28, 63.71, 63.14, 55.72, 26.04, 23.36, 10.32, 7.97 ppm; IR (KBr) 3330.5, 2960.2, 2599.6, 2456.4, 2345.0, 1951.6, 1743.3, 1496.5, 1346.1, 1110.8, 1072.2, 852.4, 700.0, 586.3 cm⁻¹; HRMS (FAB+) for C₂₆H₃₁N₄O₂S [M+H]⁺ Calcd: 463.2168, Found: 463.2186.

### (9) 1-[(1R,2R)-2-Amino-1,2-diphenylethyl]-3-(naphthalene-1-yl)thiourea (Chemical Formula 4-9)

[α]_{D}²⁵+0.157 (c 1.00, CH₃Cl); ¹H-NMR (300 MHz, DMSO-d6) δ9.93 (s, 1H), 7.22-7.99 (m, 18H), 5.51(s, 1H), 4.34 (d, J=3 Hz, 1H) ppm; ¹³C-NMR (100 MHz, DMSO-d6) δ182.70, 143.47, 142.08, 135.06, 134.65, 130.66, 128.84, 128.55, 127.69, 127.61, 127.45, 126.99, 126.32, 126.00, 123.65, 64.01, 59.88 ppm; IR (KBr) 3340.26, 3116.55, 1951.70, 1511.99, 1249.70, 941.13, 701.99, 632.56 cm⁻¹; HRMS (FAB+) for C₂₅H₂₄N₃S [M+H]⁺ Calcd: 398.1691, Found: 398.551

1.3. Asymmetric Michael Addition Reaction Between Chiton and α,β-Unsaturated Nitroalkene

1.3.1. In Case of Ketone

A thiourea catalyst (Chemical Formula 4, 7.3 mg, 0.020 mmol), 4-nitrophenol (5 mol %), and α,β-unsaturated nitroalkene (Chemical Formula 2, 30 mg, 0.20 mmol) were added to a reaction container at room temperature and dissolved in water (1.0 mL) under an air condition. Subsequently, ketone (Chemical Formula 1, however, except for R₁=H, 0.21 mL, 2.0 mmol) was added and stirred for 5 hours. After completion of the reaction with distilled water, the mixture was extracted with dichloromethane (20 mL×3 times), dehydrated with MgSO₄, filtrated, and concentrated under reduced pressure, and then a product (Chemical Formula 3) was isolated using column chromatography (SiO₂, EtOAc:hexane=5:1).

1.3.2. In Case of Aldehyde

A thiourea catalyst (Chemical Formula 4, 5 mol %) and α,β-unsaturated nitroalkene (Chemical Formula 2, 0.3 mmol) were added to a reaction container at room temperature under an air condition and dissolved in water (1.0 mL) under the air condition. Then, aldehyde (Chemical Formula 1, however, R₁=H, 5 equiv.) was added and stirred for 4 to 12 hours. After completion of the reaction with distilled water, the mixture was extracted with dichloromethane (20 mL×3 times), dehydrated with MgSO₄, filtrated, and concentrated under reduced pressure, and then a product (Chemical Formula 3) was isolated using column chromatography (SiO₂, EtOAc:hexane=5:1).

Non-limiting examples of the chiral nitro derivatives prepared by the asymmetric Michael addition reaction between the ketone or aldehyde and the α,β-unsaturated nitroalkene according to an embodiment of the present disclosure were as follows:

### (1) (S)-5-Nitro-4-phenylpentan-2-one (Chemical Formula 3-1)

[α]D²⁰+4.5 (c 1.0, CHCl₃); ¹H NMR (400 MHz, CDCl₃) δ7.35-7.21 (m, 5H), 4.72-4.67 (dd, J=12.3, 7.0 Hz, 1H), 4.63-4.58 (dd, J=12.3, 7.6 Hz, 1H), 4.05-3.98 (m, 1H), 2.92 (d, J=7.0 Hz, 2H), 2.13 (s, 3H) ppm; ¹³C NMR (100 MHz, CDCl₃) δ205.67, 138.99, 129.30, 128.14, 127.59, 79.67, 46.33, 39.23, 30.65 ppm; IR (KBr) 3064, 3035, 2968, 2948, 2919, 2902, 1714, 1548, 1384, 1361, 1163, 758, 696, 548 cm⁻¹; LRMS (FAB+) for C₁₁H₁₃NO₃ [M+H]⁺ Calcd: 208, Found: 208; HPLC [Chiralcel AD-H, hexane/2-propanol=4/96, flow rate=1.0 mL/min, λ=254 nm, retention times: (major) 28.2 min, (minor) 38.3 min];

### (2) (R)-2-[(S)-2-Nitro-1-phenylethyl]cyclohexanone (Chemical Formula 3-2)

[α]_{D}²⁶+37.5 (c 0.100, CHCl₃); ¹H NMR (300 MHz, CDCl₃) 7.34-7.23 (m, 3H), 7.16 (d, J=7 Hz, 2H), 4.95 (dd, J=12, 5 Hz, 1H), 4.63 (dd, J=12, 10 Hz, 1H), 3.76 (dt, J=10, 5 Hz, 1H), 2.68 (m, J=11, 9, 8 Hz, 1H), 2.49-2.33 (m, 2H), 2.10-2.04 (m, 1H), 1.79-1.52 (m, 4H), 1.52-1.19 (m, 1H); ¹³C NMR (100 MHz, CDCl₃) 211.9, 137.6, 128.8, 128.1, 127.7, 78.8, 52.4, 43.8, 42.7, 33.1, 28.5, 24.9, IR(KBr) 3855.1, 3650.7, 3448.2, 2921.1, 1700.9, 1552.4, 1382.7, 1243.9, 1130.1, 696.2, 561.2 cm⁻¹; LRMS(FAB+) for C₁₄H₁₇NO₃[M+H]⁺ Calcd:247, Found: 247; chiral-phase HPLC, AS-H column (i-PrOH/hexane=20:80), 254 nm, flow rate=0.5 ml/min, tr (major)=19.74 min, tr (minor)=27.57 min.

### (3) (R)-2-[(S)-2-Nitro-1-phenylethyl]cyclopentanone(Chemical Formula 3-3)

[α]_{D}²³+12.1(c 1.00, CHCl₃); ¹H NMR (300 MHz, CDCl₃) 7.34-7.23 (m, 3H), 7.20-7.15 (m, 2H), 5.37-5.30 (m, 1H), 4.71 (dd, J=13, 10 Hz, 1H), 3.76-3.65 (m, 1H), 2.44-2.31 (m, 2H), 2.19-2.06 (m, 1H) , 1.94-1.83 (m, 2H), 1.76-1.66 (m, 1H), 1.55-1.41 (m, 1H); ¹³C NMR (100 MHz, CDCl₃) 218.5, 137.6, 128.8, 127.9, 78.2, 50.4, 44.1, 38.6, 28.3, 20.2, IR (KBr) 3363.4, 2921.7, 1731.8, 1552.4, 1378.9, 1124.3 cm⁻¹; LRMS(FAB+) for C₁₃H₁₅NO₃[M+H]⁺ Calcd: 234, Found: 234; chiral-phase HPLC, AS-H column (i-PrOH/hexane=20:80), 254 nm, flow rate=0.5 ml/min, tr (major)=22.74 min, tr (minor)=30.47 min.

### (4) (S)-3-[(S)-2-Nitro-1-phenylethyl]tetrahydropyran-4-one (Chemical Formula 3-4)

[α]_{D}²³+14.4 (c 1.00, CHCl₃); ¹H NMR (300 MHz, CDCl₃) 7.37-7.26 (m, 3H), 7.21-7.16 (m, 2H), 4.96 (dd, J=12, 5 Hz, 1H), 4.64 (dd, J=12, 10 Hz, 1H), 4.20-4.10 (m, 1H), 3.87-3.68 (m, 2H), 3.70 (dd, J=11, 5 Hz, 1H), 3.27 (dd, J=11, 10 Hz, 1H), 2.93-2.83 (m, 1H), 2.73-2.61 (m, 1H), 2.60-2.53 (m, 1H); ¹³C NMR (100 MHz, CDCl₃) 207.4, 136.1, 128.9, 127.8, 78.6, 71.5, 68.9, 53.2, 42.9, 41.2, 29.6; IR (KBr) 3278.5, 2923.7, 2291.1, 1735.7, 1457.9, 1243.9, 1087.7 cm⁻¹; chiral-phase HPLC, AS-H column (i-PrOH/hexane=50:50), 254 nm, flow rate=0.5 ml/min, tr (major)=19.29 min, tr (minor)=25.53 min.

### (5) (R)-3-[(S)-2-Nitro-1-phenylethyl]tetrahydrothiopyran-4-one (Chemical Formula 3-5)

[α]_{D}²³+21.9 (c 1.00, CHCl₃); ¹H NMR (300 MHz, CDCl₃) 7.37-7.26 (m, 3H), 7.21-7.16 (m, 2H), 4.96 (dd, J=12, 5 Hz, 1H), 4.64 (dd, J=12, 10 Hz, 1H), 4.20-4.10 (m, 1H), 3.87-3.68 (m, 2H), 3.70 (dd, J=11, 5 Hz, 1H), 3.27 (dd, J=11, 10 Hz, 1H), 2.93-2.83 (m, 1H), 2.73-2.61 (m, 1H), 2.60-2.53 (m, 1H); ¹³C NMR (100 MHz, CDCl₃) 209.5, 136.5, 129.3, 128.3, 128.2, 78.6, 55.0, 44.6, 53.5, 35.1, 31.6; IR (KBr) 3367.2, 1646.9, 1376.9, 1093.4 cm⁻¹; chiral-phase HPLC, AS-H column (i-PrOH/hexane=50:50), 254 nm, flow rate=0.5 ml/min, tr (major)=18.40 min, tr (minor)=26.84 min.

### (6) (S)-4-Nitro-1,3-diphenyl-butan-1-one (Chemical Formula 3-6)

[α]_{D}²⁰−18.5 (c 1.0, CHCl₃); ¹H NMR (300 MHz, CDCl₃) δ7.91-7.92 (m, 2H), 7.59-7.26 (m, 8H), 4.85-4.81 (dd, J=12.5, 6.7 Hz, 1H), 4.71-4.67 (dd, J=12.5, 7.8 Hz, 1H), 4.26-4.20 (m, 1H), 3.51-3.46 (dd, J=17.7, 6.4 Hz, 1H), 3.45-3.40 (dd, J=17.7, 7.5 Hz, 1H) ppm; ¹³C NMR (125 MHz, CDCl₃) δ196.87, 139.15, 136.39, 133.60, 129.09, 128.77, 128.04, 127.90, 127.48, 79.58, 41.54, 39.30 ppm; IR (KBr) 3058, 3029, 2920, 1687, 1544, 1440, 1367, 1268, 1224, 1084, 988, 764, 703, 623, 559 cm⁻¹; LRMS (ESI+) for C₁₆H₁₅NO₃ [M+Na]⁺ Calcd: 292.1, Found: 292.1; HPLC [Chiralcel AD-H, hexane/2-propanol=90/10, flow rate=1.0 mL/min, λ=254 nm, retention times: (major) 12.8 min, (minor) 17.4 min];

### (7) (S)-5-Nitro-4-(p-tolyl)pentan-2-one (Chemical Formula 3-7)

[α]_{D}²⁰+4.7 (c 1.5, CHCl₃); ¹H NMR (500 MHz, CDCl₃) δ7.13-7.08 (m, 4H), 4.68-4.64 (dd, J=12.3, 6.9 Hz, 1H), 4.58-4.54 (dd, J=12.1, 7.7 Hz, 1H), 3.99-3.93 (m, 1H), 2.89 (d, J=7.7 Hz, 1H), 2.31 (s, 3H), 2.10 (s, 3H) ppm; ¹³C NMR (125 MHz, CDCl₃) δ205.57, 137.62, 135.76, 129.74, 127.24, 79.63, 46.22, 38.76, 30.41, 21.05 ppm; IR (KBr) 3056, 3029, 2976, 2944, 2924, 1717, 1551, 1378, 1365, 1163, 816, 544 cm⁻¹; LRMS (ESI+) for C₁₂H₁₅NO₃ [M+Na]⁺ Calcd: 244.1, Found: 244.2; HPLC [Chiralcel AS-H, hexane/2-propanol=80/20, flow rate=1.0 mL/min, λ=213 nm, retention times: (major) 13.1 min, (minor) 20.4 min];

### (8) (S)-4-(4-Methoxyphenyl)-5-nitropentan-2-one (Chemical Formula 3-8)

[α]_{D}²⁰−5.7 (c 1.0, CHCl₃); ¹H NMR (500 MHz, CDCl₃) δ7.13 (d, J=8.8 Hz, 2H), 6.85 (d, J=8.8 Hz, 2H), 4.67-4.63 (dd, J=12.3, 6.8 Hz, 1H), 4.57-4.53 (dd, J=12.3, 7.8 Hz, 1H), 3.98-3.92 (m, 1H), 3.78 (s, 3H), 2.88 (d, J=7.0, 2H), 2.11 (s, 3H) ppm; ¹³C NMR (125 MHz, CDCl₃) δ205.55, 159.13, 130.67, 128.45, 114.44, 79.73, 55.26, 46.29, 38.42, 30.43 ppm; IR (KBr) 3028, 3002, 2960, 2901, 1715, 1550, 1517, 1260, 1180, 1033, 813,544 cm⁻¹; LRMS (ESI+) for C₁₂H₁₅NO₄ [M+Na]⁺Calcd: 260.1, Found: 260.1; HPLC [Chiralcel AS-H, hexane/2-propanol=80/20, flow rate=1.4 mL/min, λ=213 nm, retention times: (major) 16.2 min, (minor) 37.9 min];

### (9) (S)-4-(4-Chlorophenyl)-5-nitropentan-2-one (Chemical Formula 3-9)

[α]_{D}²⁰−2.9 (c 1.0, CHCl₃); ¹H NMR (500 MHz, CDCl₃) δ7.32-7.29 (m, 2H), 7.17-7.15 (m, 2H), 4.70-4.66 (dd, J=12.4, 6.7 Hz, 1H), 4.59-4.55 (dd, J=12.4, 7.9 Hz, 1H), 4.02-3.96 (m, 1H), 2.94-2.90 (dd, J=18.5, 7.0 Hz, 1H), 2.89-2.85 (dd, J=19.0, 7.0 Hz, 1H), 2.12 (s, 3H) ppm; ¹³C NMR (125 MHz, CDCl₃) δ205.03, 137.35, 133.79, 129.26, 128.82, 79.19, 45.97, 38.40, 30.39 ppm; LRMS (ESI+) for C₁₁H₁₂ClNO₃ [M+Na]⁺ Calcd: 264.0, Found: 264.1; HPLC [Chiralcel AD-H, hexane/2-propanol=5/95, flow rate=1.0 mL/min, λ=254 nm, retention times: (major) 14.1 min, (minor) 21.9 min]; Rf (SiO₂, EtOAc/n-hexane=1/5)=0.22

### (10) (S)-4-(4-Bromophenyl)-5-nitropentan-2-one (Chemical Formula 3-10)

[α]_{D}²⁰−0.6 (c 0.4, CHCl₃); ¹H NMR (500 MHz, CDCl₃) δ7.45 (d, J=7.0 Hz, 2H), 7.10 (d, J=7.0 Hz, 2H), 4.69-4.66 (dd, J=12.5, 6.5 Hz, 1H), 4.59-4.55 (dd, J=12.7, 7.5 Hz, 1H), 4.0-3.95 (m, 1H), 2.88 (d, J=7.5 Hz, 2H), 2.12 (s, 3H) ppm; ¹³C NMR (125 MHz, CDCl₃) δ205.04, 137.91, 132.20, 129.18, 121.85, 79.10, 45.91, 38.46, 30.38 ppm; LRMS (ESI+) for CH₁₂BrNO₃ [M+Na]⁺ Calcd: 308.0, Found: 308.0; HPLC [Chiralcel AD-H, hexane/2-propanol=80/20, flow rate=1.0 mL/min, λ=210 nm, retention times: (major) 10.9 min, (minor) 12.5 min];

### (11) (S)-4-(2-Methoxyphenyl)-5-nitropentan-2-one (Chemical Formula 3-11)

[α]_{D}²⁰+16.4 (c 1.6, CHCl₃); ¹H NMR (500 MHz, CDCl₃) δ7.26-7.23 (m, 1H), 7.15-7.13 (m, 1H), 6.92-6.87 (m, 2H), 4.76-4.69 (m, 2H), 4.25-4.19 (m, 1H), 3.86 (s, 3H), 3.05-3.0 (dd, J=18.0, 7.5 Hz, 1H), 2.98-2.93 (dd, J=18.0, 7.0 Hz, 1H), 2.13 (s, 3H) ppm; ¹³C NMR (125 MHz, CDCl₃) δ206.16, 157.09, 129.31, 129.01, 126.44, 120.94, 110.99, 77.85, 55.35, 44.52, 35.33, 30.25 ppm; LRMS(ESI+) for C₁₂H₁₅NO₄[M+Na] Calcd: 260.1, Found: 260.1; HPLC [Chiralcel AD-H, hexane/2-propanol=5/95, flow rate=1.0 mL/min, λ=254 nm, retention times: (major) 14.1 min, (minor) 21.9 min];

### (12) (S)-4-(Furan-2-yl)-5-nitropentan-2-one (Chemical Formula 3-12)

[α]_{D}²⁰−8.1 (c 1.0, CHCl₃); ¹H NMR (500 MHz, CDCl₃) δ7.34-7.33 (dd, J=1.8, 0.7 Hz, 1H), 6.30-6.29 (dd, J=3.2, 1.8 Hz, 1H), 6.14 (d, J=3.2 Hz, 1H), 4.71-4.68 (dd, J=12.6, 6.6 Hz, 1H), 4.67-4.64 (dd, J=12.6, 6.6 Hz, 1H), 4.13-4.07 (m, 1H), 3.00-2.95 (dd, J=16.7, 5.2 Hz, 1H), 2.93-2.87 (dd, J=16.7, 6.0 Hz, 1H), 2.18 (s, 3H) ppm; ¹³C NMR (125 MHz, CDCl₃) δ205.11, 151.69, 142.31, 110.50, 107.10, 77.07, 43.49, 32.89, 30.22 ppm; IR (flim) 3151, 3124, 2920, 1714, 1552, 1430, 1377, 1165, 1015, 741 cm⁻¹; LRMS (ESI+) for C₉H₁₁NO₄ [M+Na]⁺ Calcd: 220.1, Found: 220.1; HPLC [Chiralcel AD-H, hexane/2-pro panol=94/6, flow rate=1.0 mL/min, λ=213 nm, retention times: (major) 10.5 min, (minor) 11.6 min];

### (13) (R)-2-[(S)-1-(3-Methylphenyl)-2-nitroethyl]cyclohexanone (Chemical Formula 3-13)

[α]_{D}²³+10.8 (c 1.00, CHCl₃); ¹H NMR (300 MHz, CDCl₃) 7.12 (d, J=8 Hz, 2H), 7.04 (d, J=8 Hz, 2H), 4.91 (dd, J=12, 5 Hz, 1H), 4.60 (dd, J=12, 10 Hz, 1H), 3.72 (dt, J=10, 5 Hz, 1H), 2.71-2.62 (m, 1H), 2.50-2.37 (m, 2H), 2.10 (s, 3H), 2.08 (m, 1H), 1.81-1.53 (m, 5H); ¹³C NMR (75 MHz, CDCl₃) δ212.0, 137.4, 134.5, 129.5, 127.9, 78.9, 52.5, 43.5, 42.7, 33.1, 28.5, 24.9, 21.0; IR (KBr) 3286.2, 2925.6, 1704.8, 1552.4, 13789, 1130.1, 819.6 cm⁻¹; LRMS(FAB+) for C₁₅H₁₉NO₃ [M+H]⁺ Calcd: 262, Found: 262 chiral-phase HPLC, AD-H column (i-PrOH/hexane=5:95), 254 nm, flow rate=1 ml/min, tr (major)=10.54 min, tr (minor)=12.98 min.

### (14) (R)-2-[(S)-1-(4-Isopropylphenyl)-2-nitroethyl]cyclohexanone (Chemical Formula 3-14)

[α]_{D}²³+16.9 (c 1.00, CHCl₃); ¹H NMR (300 MHz, CDCl₃) 7.16 (d, J=8 Hz, 2H), 7.07 (d, J=8 Hz, 2H), 4.91 (dd, J=12, 4 Hz, 1H), 4.62 (dd, J=12, 10 Hz, 1H), 3.76-3.70 (m, 1H), 2.88-2.85 (m, 1H), 2.71-2.65 (m, 1H), 2.49-2.38 (m, 2H), 2.09-2.04 (m, 1H), 1.81-1.58 (m, 5H), 1.22 (d, J=7 Hz, 6H); ¹³C NMR (75 MHz, CDCl₃) 212.2, 148.2, 134.8, 127.9, 126.9, 78.9, 52.5, 43.4, 33.6, 29.6, 28.5, 24.9, 23.8; IR (KBr) 3853.2, 3357.6, 2925.6, 2345.1, 1706.7, 1552.4, 1378.9, 1089.6 cm⁻¹, chiral-phase HPLC, AD-H column (i-PrOH/hexane=3:97), 254 nm, flow rate=1 ml/min, tr (major)=10.19 min, tr (minor)=12.15 min.

### (15) (R)-2-[(S)-1-(4-Fluorophenyl)-2-nitroethyl]cyclohexanone (Chemical Formula 3-15)

[α]_{D}²³+29.2 (c 1.00, CHCl₃); ¹H NMR (300 MHz, CDCl₃) 7.14-7.17 (m, 2H), 7.03-6.99 (m, 2H), 4.95 (dd, J=8, 5 Hz, 1H), 4.59 (dd, J=12, 10 Hz, 1H), 3.80-3.74 (m, 1H), 2.67-2.65 (m, 1H), 2.49-2.32 (m, 2H), 2.12-2.04 (m, 1H), 1.81-1.56 (m, 4H), 1.26-1.20 (m, 1H); ¹³C NMR (75 MHz, CDCl₃) 211.6, 163.3, 160.8, 133.3, 129.7, 115.9, 78.8, 52.5, 43.2, 42.7, 33.1, 28.4, 25.0; IR (KBr) 3266.9, 2923.7, 2279.5, 1708.7, 1550.5, 1376.9, 1093.4, 853.0 cm⁻¹; LRMS(FAB+) for C₁₄H₁₆FNO₃ [M+H]⁺ Calcd: 266, Found: 266; chiral-phase HPLC, AS-H column (i-PrOH/hexane=10:90), 254 nm, flow rate=1 ml/min, tr (major)=17.62 min, tr (minor)=23.07 min.

### (16) (R)-2-[(S)-1-(4-Chlorophenyl)-2-nitroethyl]cyclohexanone (Chemical Formula 3-16)

[α]_{D}²³+24.6 (c 1.00, CHCl₃); ¹H NMR (300 MHz, CDCl₃) 7.31 (d, J=8 Hz, 2H), 7.13 (d, J=8 Hz, 2H), 4.96 (dd, J=12, 4 Hz, 1H), 4.62 (dd, J=12, 10 Hz, 1H), 3.78 (dt, J=10, 4 Hz, 1H), 2.69-2.60 (m, 1H), 2.51-2.34 (m, 2H), 2.15-2.05 (m, 1H), 1.83-1.52 (m, 4H), 1.28-1.23 (m, 1H), ¹³C NMR (75 MHz, CDCl₃) 211.6, 136.3, 133.7, 129.6, 129.2, 78.6, 52.4, 43.4, 42.8, 33.2, 28.5, 25.1; IR (KBr) 3252.2, 2897.7, 1721.7, 1555.0, 1087.5 cm⁻¹; chiral-phase HPLC, AS-H column (i-PrOH/hexane=10:90), 254 nm, flow rate=1 ml/min, tr (major)=14.96 min, tr (minor)=21.15 min.

### (17) (R)-2-[(S)-1-(4-Bromophenyl)-2-nitroethyl]cyclohexanone (Chemical Formula 3-17)

[α]_{D}²⁵+16.4 (c 0.80, CHCl₃); ¹H NMR (300 MHz, CDCl₃) 7.46 (d, J=8 Hz, 2H), 7.06 (d, J=8 Hz, 2H), 4.93 (dd, J=12, 4 Hz, 1H), 4.60 (dd, J=12, 10 Hz, 1H), 3.75 (dt, J=10, 4 Hz, 1H), 2.69-2.60 (m, 1H), 2.51-2.32 (m, 2H), 2.15-2.05 (m, 1H), 1.85-1.58 (m, 4H), 1.30-1.16 (m, 1H); ¹³C NMR (75 MHz, CDCl₃) 211.4, 136.7, 132.0, 129.8, 121.7, 78.4, 52.3, 43.4, 42.7, 33.1, 28.4, 25.0; IR (KBr) 3373.0, 2927.5, 1706.7, 1550.5, 1376.9, 1087.7, 827.3 cm⁻¹; LRMS(FAB+) for C₁₄H₁₆BrNO₃ [M+H]⁺ Calcd: 326, Found: 326; chiral-phase HPLC, AS-H column (i-PrOH/hexane=10:90), 254 nm, flow rate=0.5 ml/min, tr (major)=20.22 min, tr (minor)=29.08 min.

### (18) (R)-2-[(S)-1-(3-Chlorophenyl)-2-nitroethyl]cyclohexanone (Chemical Formula 3-18)

[α]_{D}²³+17.2 (c 1.00, CHCl₃); ¹H NMR (300 MHz, CDCl₃) 7.26-7.24 (m, 2H), 7.17 (s, 1H), 7.09-7.06 (m, 1H), 4.94 (dd, J=13, 4 Hz, 1H), 4.61 (d, J=13, 10 Hz, 1H), 3.75 (dt, J=10, 4 Hz, 1H), 2.69-2.64 (m, 1H), 2.62-2.34 (m, 2H), 2.13-2.07 (m, 1H), 1.82-1.57 (m, 4H), 1.23-1.19 (m, 1H); ¹³C NMR (75 MHz, CDCl₃) 211.4, 139.8, 134.7, 130.1, 128.2, 128.0, 126.4, 78.3, 52.2, 43.6, 42.7, 33.2, 28.4, 25.0; IR (KBr) 3284.3, 2929.4, 2362.4, 1706.7, 1552.4, 1378.9, 1130.1, 794.5 cm⁻¹; chiral-phase HPLC, AS-H column (i-PrOH/hexane=10:90), 254 nm, flow rate=1 ml/min, tr (major)=16.09 min, tr (minor)=22.16 min.

### (19) (R)-2-[(R)-1-(Furan-2-yl)-2-nitroethyl]cyclohexanone (Chemical Formula 3-19)

[α]_{D}²³+12.4 (c 2.00, CHCl₃); ¹H NMR (300 MHz, CDCl₃) 7.35 (m, 1H), 6.28 (dd, J=3, 2 Hz, 1H), 6.18 (d, J=3 Hz, 1H), 4.83-4.63 (m, 2H), 3.97 (dt, J=10, 5 Hz, 1H), 2.80-2.71 (m, 1H), 2.50-2.31 (m, 2H), 2.14-2.04 (m, 1H), 1.88-1.56 (m, 4H), 1.35-1.21 (m, 1H); ¹³C NMR (75 MHz, CDCl₃) 211.0, 150.9, 142.3, 110.3, 109.0, 77.1, 51.1, 42.6, 37.6, 32.5, 28.2, 25.1; IR (KBr) 3392., 2941.0, 1706.7, 1552.4, 1430.9, 1376.9, 1130.1, 1014.4, 916.0, 740.5 cm⁻¹; HRMS(FAB+) for C₁₂H₁₅NO₄ [M+H]⁺ Calcd: 238.1079, Found: 238.1080; chiral-phase HPLC, AD-H column (i-PrOH/hexane=5:95), 254 nm, flow rate=0.7 ml/min, tr (minor)=26.50 min, tr (major)=33.60 min.

### (20) (R)-2-[(S)-1-Thien-2-yl-2-nitroethyl]cyclohexanone (Chemical Formula 3-20)

[α]_{D}²³+20.1 (c 1.00, CHCl₃); ¹H NMR (300 MHz, CDCl₃) 7.22-7.21 (m, 1H), 6.93 (dd, J=5, 3 Hz, 1H), 6.88-6.87 (m, 1H), 4.89 (dd, J=12, 5 Hz, 1H), 4.65 (dd, J=12, 9 Hz, 1H), 4.13 (dt, J=9, 5 Hz, 1H), 2.73-2.64 (m, 1H), 2.50-2.32 (m, 2H), 2.14-2.06 (m, 1H), 1.95-1.82 (m, 2H), 1.69-1.58 (m, 2H), 1.39-1.29 (m, 1H); ¹³C NMR (75 MHz, CDCl₃) 3392.3, 3110.7, 2939.1, 2285.3, 1704.8, 1552.4, 1378.9, 1253.5, 1128.2, 850.4, 705.8 cm⁻¹; HRMS(FAB+) for C₁₂H₁₅NO₃S [M+H]⁺ Calcd: 254.0851, Found: 254.0853; chiral-phase HPLC, AS-H column (i-PrOH/hexane=4:96), 254 nm, flow rate=0.5 ml/min, tr (minor)=29.95 min, tr (major)=35.16 min.

### (21) (R)-2-[(S)-1-Naphthalen-2-yl-2-nitroethyl]cyclohexanone (Chemical Formula 3-21)

[α]_{D}²³+15.6 (c 1.00, CHCl₃); ¹H NMR (300 MHz, CDCl₃) 7.84-7.78 (m, 3H), 7.64 (s, 1H), 7.50-7.46 (m, 2H), 7.29 (dd, J=11, 2 Hz, 1H), 5.03 (dd, J=12, 4 Hz, 1H), 4.74 (dd, J=12, 10 Hz, 1H), 3.95 (dt, J=10, 4 Hz, 1H), 2.83-2.74 (m, 1H), 2.54-2.36 (m, 2H), 2.11-2.04 (m, 1H), 1.79-1.62 (m, 4H), 1.32-1.29 (m, 1H); ¹³C NMR (75 MHz, CDCl₃) 211.8, 135.0, 133.2, 128.8, 127.7, 126.4, 126.1, 125.1, 78.8, 52.4, 44.0, 42.7, 33.3, 28.5, 25.0; IR (KBr) 3376.9, 2923.7, 1700.9, 1550.5, 1382.7, 1091.5, 825.4 cm⁻¹; LRMS(FAB+) for C₁₈H₁₉NO₃ [M]⁺ Calcd: 297, Found: 297; chiral-phase HPLC, ASH column (i-PrOH/hexane=50:50), 254 nm, flow rate=0.7 ml/min, tr (major)=9.14 min, tr (minor)=13.12 min.

### (22) (R)-2-[(S)-1-(4-Methoxyphenyl)-2-nitroethyl]cyclohexanone (Chemical Formula 3-22)

[α]_{D}²³+10.8 (c 1.00, CHCl₃); ¹H NMR (300 MHz, CDCl₃) 7.08 (d, J=9 Hz, 2H), 6.85 (d, J=9 Hz, 2H), 4.91 (dd, J=12, 4 Hz, 1H), 4.58 (dd, J=12, 10 Hz, 1H), 3.78 (s, 3H), 3.71 (dt, J=10, 4 Hz, 1H), 2.69-2.60 (m, 1H), 2.52-2.32 (m, 2H), 2.13-2.03 (m, 1H), 1.83-1.51 (m, 4H), 1.30-1.16 (m, 1H); ¹³C NMR (75 MHz, CDCl₃) 212.2, 15809, 129.4, 129.1, 114.2, 79.0, 55.1, 52.6, 43.1, 42.7, 33.1, 28.5, 24.9; IR (KBr) 3380.7, 2950.7, 2360.5, 1700.9, 1552.4, 1388.5, 1255.4, 1089.6, 831.2 cm⁻¹; HRMS(FAB+) for C₁₅H₁₉NO₄ [M]⁺ Calcd: 278.1392, Found: 278.1390; chiral-phase HPLC, AD-H column (i-PrOH/hexane=20:80), 254 nm, flow rate=0.5 ml/min, tr (major)=18.59 min, tr (minor)=23.72 min.

### (23) (R)-2-[(S)-1-(2-Methoxyphenyl)-2-nitroethyl]cyclohexanone (Chemical Formula 3-23)

[α]_{D}²³+7.2 (c 0.10, CHC₁₃); ¹H NMR (300 MHz, CDCl₃) 7.28-7.22 (m, 1H), 7.09 (dd, J=7, 2 Hz, 1H), 6.91-6.86 (m 2H), 4.84 (dd, J=10, 5 Hz, 1H), 4.00-3.90 (m, 1H), 3.84 (s, 3H), 2.99 (dt, J=10, 5 Hz, 1H), 2.50-2.34 (m, 2H), 2.10-2.04 (m, 1H), 1.80-1.54 (m, 4H), 1.12-1.18 (m, 1H); ¹³C NMR (75 MHz, CDCl₃) 212.5, 157.5, 130.9, 128.8, 125.2, 120.8, 110.9, 76.6, 55.3, 50.5, 42.6, 33.2, 28.5, 25.1; IR (KBr) 3293.9, 2935.2, 2345.1, 1706.7, 1550.5, 1378.9, 1245.8, 1122.4, 755.9 cm⁻¹; HRMS(FAB+) for C₁₅H₁₉NO₄ [M+H]⁺ Calcd: 278.1392, Found: 278.1390; chiral-phase HPLC, AS-H column (i-PrOH/hexane=10:90), 254 nm, flow rate=0.5 ml/min, tr (major)=26.35 min, tr (minor)=36.10 min.

### (24) (R)-2-[(S)-2-Nitro-1-(2-nitrophenyl)ethyl)cyclohexanone (Chemical Formula 3-24)

[α]_{D}²³+9.8 (c 0.80, CHCl₃); ¹H NMR (300 MHz, CDCl₃) 7.83 (dd, J=8, 2 Hz, 1H), 7.64-7.58 (m, 1H), 7.49-7.41 (m, 2H), 4.98-4.83 (m, 2H), 4.38 (dt, J=9, 4 Hz, 1H), 2.98-2.87 (m, 1H), 2.50-2.34 (m, 2H), 2.17-2.07 (m, 1H), 1.85-1.61 (m, 5H); ¹³C NMR (75 MHz, CDCl₃) 211.1, 150.7, 133.1, 132.8, 129.1, 128.6, 124.9, 77.6, 52.1, 42.8, 38.6, 33.2, 28.3, 25.3; IR (film) 2944, 2864, 1707, 1552, 1527, 1358, 855, 781 cm⁻¹; HRMS(FAB+) for C₁₄H₁₆N_{205 [}M+H]⁺ Calcd: 293.1137, Found: 293.1141; chiral-phase HPLC, AD-H column (i-PrOH/hexane=15:85), 254 nm, flow rate=1 ml/min, tr (major)=14.45 min, tr (minor)=22.22 min.

### (25) (R)-2,2,-Dimethyl-4-nitro-3-phenylbutanal (Chemical Formula 3-25)

[α]_{D}²⁰+11.26 (c 0.3, CH₂Cl₂); ¹H-NMR (300 MHz, CDCl₃) δ9.50 (s, 1H), 7.18-7.34 (m, 5H), 4.81-4.89 (dd, J=9.0, 12 Hz, 1H), 4.66-4.71 (dd, J=3.0, 12 Hz, 1H), 3.76-3.81 (dd, J=3.0 12 Hz, 1H) 1.10 (s, 3H), 0.97 (s, 3H) ppm; ¹³C-NMR (100 MHz, CDCl₃) δ204.25, 135.30, 129.06, 128.71, 128.15, 76.30, 48.45, 48.22, 21.70, 18.88 ppm; IR(neat) 3033, 2974, 2933, 2819, 2721, 1725, 1555, 1496, 1455, 1379, 882, 750, 705 cm⁻¹; HRMS (FAB+) for C₁₂H₁₅NO₃ [M+Na]⁺ Calcd: 244.0950 Found: 244.0950; HPLC [Chiralcel ODH, hexane/2-propanol=80/20, flow rate=0.7 mL/min, λ=254 nm, retention times: (major) 18.3 min, (minor) 31.1 min].

### (26) (R)-3-(4-Chlorophenyl)-2,2-dimethyl-4-nitrobutanal (Chemical Formula 3-26)

[α]_{D}²²−5.83 (c 0.2, CH₂Cl₂); ¹H-NMR (300 MHz, CDCl₃) δ9.50 (s, 1H), 7.31 (d, J=6.0 Hz, 2H), 7.15 (d, J=6.0 Hz, 2H), 4.79-4.85 (dd, J=6.0, 9.0 Hz, 1H), 4.67-4.71 (dd, J=3.0, 9.0 Hz, 1H), 3.75-3.79 (dd, J=3.0, 9.0 Hz, 1H), 1.12 (s, 3H), 1.00 (s, 3H) ppm; ¹³C-NMR (100 MHz, CDCl₃) δ204.07, 134.34, 134.20, 130.61, 129.16, 76.35, 48.39, 48.07, 21.95, 19.10 ppm; IR (KBr) 2926, 1728, 1556, 1494, 1378, 1094, 835 cm⁻¹; HRMS (FAB+) for C₁₂H₁₄ClNO₃ [M+Na]⁺ Calcd: 278.0560 Found: 278.0562; HPLC [Chiralcel OD-H, hexane/2-propanol=80/20, flow rate=0.7 mL/min, λ=254 nm, retention times: (major) 16.1 min, (minor) 25.4 min].

### (27) (R)-3-(4-Bromophenyl)2,2-dimethyl-4-nitrobutanal (Chemical Formula 3-27)

[α]_{D}²³−14.86 (c 0.1, CH₂Cl₂); ¹H-NMR (300 MHz, CDCl₃) δ9.49 (s, 1H), 7.47 (d, J=6.0 Hz, 2H), 7.09 (d, J=6.0 Hz, 2H), 4.79-4.85 (dd, J=9.0, 9.0 Hz, 1H), 4.67-4.71 (dd, J=3.0, 9.0 Hz, 1H), 3.74-3.78 (dd, J=3.0, 9.0 Hz, 1H), 1.12 (s, 3H), 1.01 (s, 3H) ppm; ¹³C-NMR (100 MHz, CDCl₃) δ204.04, 134.73, 132.12, 130.95, 122.49, 76.28, 48.33, 48.15, 21.98, 19.11 ppm; IR (KBr) 2924, 2857, 1727, 1555, 1457, 1377, 1009 cm⁻¹; HRMS (FAB+) for C₁₂H₁₄BrNO₃ [M+Na]⁺ Calcd: 322.0055 Found: 322.0053; HPLC [Chiralcel OD-H, hexane/2-propanol=80/20, flow rate=0.7 mL/min, λ=254 nm, retention times: (major) 21.9 min, (minor) 31.7 min].

### (28) (R)-2,2-Dimethyl-4-nitro-3-p-tolylbutanal (Chemical Formula 3-28)

[α]_{D}²²−15.07 (c 0.4, CH₂Cl₂); ¹H-NMR (300 MHz, CDCl₃) δ9.50 (s, 1H), 7.05-7.12 (m, 4H), 4.78-4.84 (dd, J=9.0, 12.0 Hz, 1H), 4.63-4.67 (dd, J=3.0, 9.0 Hz, 1H) 2.29 (s, 3H), 1.09 (s, 3H), 0.97 (s, 3H); ¹³C-NMR (100 MHz, CDCl₃) δ204.65, 138.07, 132.38, 129.60, 129.13, 76.60, 48.46, 48.34, 21.75, 21.23, 19.05 ppm; IR (KBr) 2973, 2925, 1726, 1556, 1516, 1381, 1120, 824 cm⁻¹; HRMS (FAB+) for C₁₃H₁₇NO₃ [M+Na]⁺ Calcd: 258.1106 Found: 258. 1107; HPLC [Chiralcel OD-H, hexane/2-propanol=80/20, flow rate=0.7 mL/min, λ=254 nm, retention times: (major) 14.2 min, (minor) 21.1 min].

### (29) (R)-3-(Furan-2-yl)-2,2-dimethyl-4-nitrobutanal (Chemical Formula 3-29)

[α]_{D}²²−12.06 (c 0.3, CH₂Cl₂); ¹H-NMR (300 MHz, CDCl₃) δ9.51 (s, 1H), 7.37 (d, J=0.6 Hz, 1H), 6.31-6.32 (dd, J=3.0, 3.3 Hz, 1H), 6.23 (d, J=3.0 Hz, 1H), 4.73-4.79 (dd, J=9.0, 12 Hz, 1H), 4.58-4.62 (dd, J=3.0, 9.0 Hz, 1H), 3.91-3.95 (dd, J=3.0, 9.0 Hz, 1H), 1.17 (s, 3H), 1.04 (s, 3H) ppm; ¹³C-NMR (100 MHz, CDCl₃) δ203.76, 150.02, 142.92, 110.62, 109.82, 75.09, 48.36, 42.39, 21.31, 19.21 ppm; IR (KBr) 2925, 1728, 1557, 1376, 1148, 740 cm⁻¹; HRMS (FAB+) for C₁₀H₁₃NO+[M+Na]⁺ Calcd: 234.0742 Found: 234.0742; HPLC [Chiralcel OD-H, hexane/2-propanol=90/10, flow rate=0.7 mL/min, λ=254 nm, retention times: (major) 14.7 min, (minor) 21.2 min].

### (30) (R)-3-(4-Methoxyphenyl)-2,2-dimethyl-4-nitrobutanal (Chemical Formula 3-30)

[α]_{D}²²−9.55 (c 0.2, CH₂Cl₂); ¹H-NMR (300 MHz, CDCl₃) δ9.51 (s, 1H), 7.12 (d, J=9.0 Hz, 2H), 6.85 (d, J=6.0, 2H), 4.78-4.84 (dd, J=9.0, 12 Hz, 1H), 4.64-4.68 (dd, J=3.0, 9.0 Hz, 1H), 3.77 (s, 3H), 3.71-3.75 (dd, J=3.0, 9.0 Hz, 1H), 1.11 (s, 3H), 0.98 (s, 3H) ppm; ¹³C-NMR (100 MHz, CDCl₃) 8204.73, 159.47, 130.32, 127.30, 114.26, 76.69, 55.41, 48.57, 47.98, 21.71, 19.10 ppm; IR (KBr) 2927, 1725, 1611, 1556, 1514, 1465, 1380, 1252, 1033, 836 cm⁻¹; HRMS(FAB+) for C₁₃H₁₇NO+[M+Na]⁺ Calcd: 274.1055 Found: 274.1055; HPLC [Chiralcel OD-H, hexane/2-propanol=80/20, flow rate=0.7 mL/min, λ=254 nm, retention times: (major) 18.2 min, (minor) 25.7 min].

### (31) (R)-3-(2-Methoxyphenyl)-2,2-dimethyl-4-nitrobutanal (Chemical Formula 3-31)

[α]_{D}²⁰−14.39 (c 0.3, CH₂Cl₂); ¹H-NMR (300 MHz, CDCl₃) δ9.50 (s, 1H), 7.23-7.29 (m, 1H), 7.11-7.14 (dd, J=3.0, 9.0 Hz, 1H), 6.87-6.95 (m, 2H), 4.86-4.94 (dd, J=12, 15 Hz, 1H), 4.69-4.75 (dd, J=3.0, 12 Hz, 1H), 4.22 (d, J=9.0 Hz, 1H), 3.81 (s, 3H), 1.09 (s, 3H), 1.05 (s, 3H) ppm; ¹³C-NMR (100 MHz, CDCl₃) δ204.13, 157.36, 129.79, 129.28, 124.00, 120.76, 111.29, 75.81, 55.34, 48.37, 21.00, 19.96 ppm; HRMS (FAB+) for C₁₃H₁₇NO₄ [M+Na]⁺ Calcd: 274.1055 Found: 274.1056; IR (KBr) 2927, 1725, 1611, 1556, 1514, 1465, 1380, 1252, 1033, 836 cm⁻¹; HPLC [Chiralcel OD-H, hexane/2-propanol=90/10, flow rate=0.7 mL/min, λ=254 nm, retention times: (major) 15.4 min, (minor) 25.2 min].

### (32) (R)-3-(4-Hydroxyphenyl)-2,2-dimethyl-4-nitrobutanal (Chemical Formula 3-32)

[α]_{D}²²−13.37 (c 0.2, CH₂Cl₂); ¹H-NMR (300 MHz, CDCl₃) δ9.52(s, 1H), 7.04-7.07 (m, 2H), 6.74-6.77 (m, 2H), 5.47 (br s, 1H), 4.77-4.85 (dd, J=12, 12 Hz, 1H), 4.64-4.69 (dd, J=3.0, 12.0 Hz, 1H), 3.69-3.75 (dd, J=6.0, 12.0 Hz), 1.12 (s, 3H), 1.00 (s, 3H) ppm; ¹³C-NMR (100 MHz, CDCl₃) 8205.71, 155.80, 130.47, 127.07, 115.8, 76.71, 48.67, 48.01, 21.40, 19.07 ppm; IR (KBr) 2925, 1726, 1638, 1556, 1456, 1380, 705 cm⁻¹; HRMS (FAB+) for C₁₂H₁₅NO₄ [M+Na]⁺ Calcd: 260.0899 Found: 260.0899; HPLC [Chiralcel ODH, hexane/2-propanol=80/20, flow rate=0.7 mL/min, λ=254 nm, retention times: (major) 16.1 min, (minor) 24.9 min].

### (33) (2R,3S)-2-Methyl-4-nitro-3-phenylbutanal (Chemical Formula 3-33)

[α]_{D}²⁰−9.00 (c 0.2, CH₂Cl₂); ¹H-NMR (300 MHz, CDCl₃) δ9.71 (s, 1H), 7,15-7.36 (m, 5H), 4.77-4.83 (dd, J=6.0, 12 Hz, 1H), 4.64-4.71 (dd, J=9.0, 12 Hz, 1H), 3.77-3.87 (m, 1H), 2.72-2.83 (m, 1H), 1.00 (d, J=9.0 Hz, 3H) ppm; ¹³C-NMR (100 MHz, CDCl₃) δ202.53, 136.75, 129.30, 128.35, 128.28, 78.33, 48.65, 44.24, 12.36 ppm; IR (neat) 3031, 2975, 2933, 2827, 2729, 1724, 1603, 1551, 1496, 1433, 1459, 1380, 1203, 914, 853, 757, 702 cm⁻¹; LRMS (FAB+) for C₁H₁₃NO₃ [M+Na]⁺ Calcd: 230.1 Found: 230.1; HPLC [Chiralcel OD-H, hexane/2-propanol=90/10, flow rate=1.0 mL/min, λ=254 nm, retention times: (major) 35.4 min, (minor) 25.4 min].

### (34) (2R,3S)-2-Ethyl-4-nitro-3-phenylbutanal (Chemical Formula 3-34)

[α]_{D}²⁰+8.10 (c 0.4, CH₂Cl₂); ¹H-NMR (300 MHz, CDCl₃) δ9.72 (d, J=3.0 Hz, 1H), 7.28-7.35 (m, 3H), 7.16-7.20 (m, 2H), 4.74-4.81 (dd, J=9.0, 18 Hz), 4.63-4.71 (dd, J=3.0, 15 Hz), 3.75-3.83 (m, 1H), 2.64-2.72 (m, 1H), 1.46-1.56 (m, 2H), 0.83 (t, J=9.0 Hz, 3H) ppm; ¹³C-NMR (100 MHz, CDCl₃) 8203.32, 136.75, 129.11, 128.21, 127.99, 78.55, 54.98, 42.68, 20.36, 10.66 ppm; IR (neat) 3031, 2969, 2878, 2738, 1719, 1551, 1496, 1456, 1432, 1379, 1206, 757, 702 cm⁻¹; LRMS (FAB+) for C₁₂H₁₅NO₃ [M+Na]⁺ Calcd: 244.1 Found: 244.1; HPLC [Chiralcel OD-H, hexane/2-propanol=90/10, flow rate=0.8 mL/min, λ=215 nm, retention times: (major) 34.5 min, (minor) 29.9 min].

### (35) (R)-2-[(S)-2-Nitro-1-phenylethyl]pentanal (Chemical Formula 3-35)

[α]_{D}²⁰+15.40 (c 0.2, CH₂Cl₂); ¹H-NMR (300 MHz, CDCl₃) δ9.70 (d, J=3.0 Hz, 1H), 7.27-7.37 (m, 3H), 7.16-7.19 (m, 3H), 4.60-4.81 (m, 2H), 3.73-3.81 (m, 3H), 2.60-2.74 (m, 3H), 1.12-1.55 (m, 4H), 0.79 (t, J=6.0 Hz, 3H) ppm; ¹³C-NMR (100 MHz, CDCl₃) δ203.28, 136.77, 129.11, 128.22, 127.98, 78.42, 53.78, 43.13, 29.44, 19.75, 13.93 ppm; IR (neat) 2968, 1719, 1553, 1379 cm⁻¹; LRMS (FAB+) for C₁₃H₁₇NO₃ [M+Na]⁺ Calcd: 258.1 Found: 258.1; HPLC [Chiralcel OD-H, hexane/2-propanol=80/20, flow rate=1.0 mL/min, λ=254 nm, retention times: (major) 19.3 min, (minor) 14.1 min].

### (36) (2R,3S)-2-Isopropyl-4-nitro-3-phenylbutanal (Chemical Formula 3-36)

[α]_{D}²⁰+19.81 (c 0.5, CH₂Cl₂); ¹H-NMR (300 MHz, CDCl₃) δ9.93 (d, J=1.8 Hz, 1H), 7.28-7.37 (m, 3H), 7.15-7.20 (m, 2H), 4.65-4.69 (dd, J=3.0, 9.0 Hz, 1H), 4.55-4.60 (dd, J=6.0, 9.0 Hz, 1H), 3.87-3.93 (m, 1H), 2.75-2.80 (m, 1H), 1.68-1.76 (m, 1H), 1.11 (d, J=6.0 Hz, 3H), 0.89 (d, J=6.0 Hz, 3H) ppm; ¹³C-NMR (100 MHz, CDCl₃) δ204.59, 137.26, 129.37, 128.32, 128.16, 79.21, 58.95, 42.13, 28.13, 21.88, 17.18 ppm; IR (neat) 2964, 1717, 1553, 1379 cm⁻¹; HRMS (FAB+) for C₁₃H₁₇NO₃ [M+H]⁺ Calcd: 236.1208 Found: 236.1287; HPLC [Chiralcel AD-H, hexane/2-propanol=99.5/0.5, flow rate=0.3 mL/min, λ=254 nm, retention times: (major) 24.1 min, (minor) 29.6 min]

### EXAMPLE 2

### Synthesis of chiral indole Derivatives of Present Disclosure

A solution of (R)-2-((S)-1-(4-methoxyphenyl)-2-nitroethyl)cyclohexan-1-one (Chemical Formula 3-22, 300 mg, 1.08 mmol) in THF (5 mL) was added with zinc powder (707 mg, 10.8 mmol) and added with a solution of NH₄Cl (57 mg, 1.08 mmol) in water (2 mL). The mixture was vigorously stirred at room temperature for 6 hours and filtered. A residual solid was washed with THF and the combined filtrate was concentrated. The residue was purified by flash column chromatography (CH₂Cl₂/MeOH, 95/5) on a silica gel to obtain 197 mg (95%) of 3-arylhexahyroindole 1-oxides (Chemical Formula 5-1) as a white solid (Reaction Formula 3).

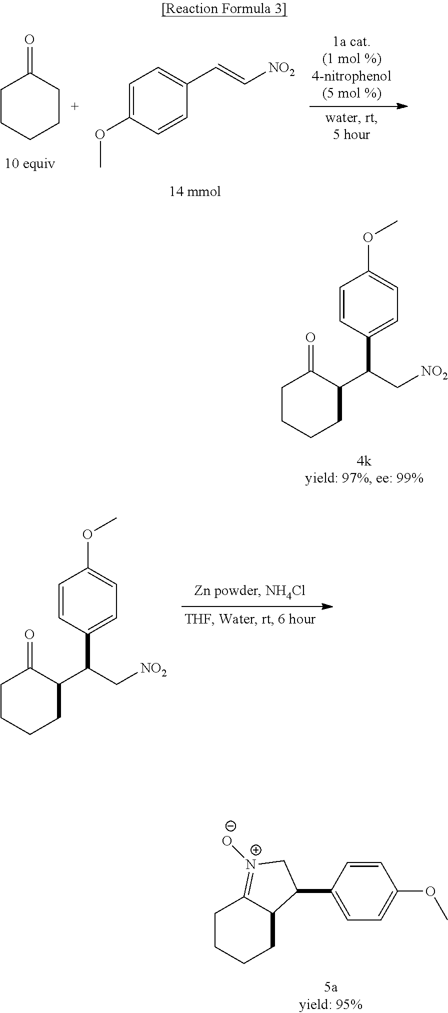

### (3R,3aS)-3-(4-Methoxyphenyl)-3,3a,4,5,6,7-hexahydro-2H-indole-1-Oxide (Chemical Formula 5-1)

¹H NMR (500 MHz, CDCl₃): 7.16 (d, 2H, J=8.7, ArH), 6.89 (d, 2H, J=8.7, ArH), 4.27-4.24 (br m, 1H, NCH₂), 4.15-4.10 (br m, 1H, NCH2), 3.81 (s, 3H, OCH₃), 3.25-3.15 (m, 2H, ArCH, NCCH2), 2.8-2.7 (ArCHCH), 2.12-1.84 (m, 3H, CH₂), 1.85 (br d, 1H, J=12.8, CH₂), 1.45-1.18 (m, 4H, CH₂); ¹³C NMR (125 MHz, CDCI3): δ158.9, 148.6, 131.7, 128.3, 114.4, 68.4, 55.3, 50.6, 45.3, 32.3, 24.3, 23.8, 23.5; LRMS(ESI+): m/z 246.1 (M+H);

In the presence of zinc powder, since a chiral nitro derivative (Chemical Formula 3-22) was easily hydrogenated to the corresponding chiral indole derivative to exhibit a yield of 95%, the reaction was potentially very useful in association with organic synthesis for forming carbon-carbon bonds.

### EXPERIMENTAL EXAMPLE 1

### Optimization of thiourea catalyst

In order to examine an effect of a catalyst on an enantioselective Michael addition reaction of nitroalkene and aldehyde, the reaction was performed using isobutyraldehyde and trans-beta-nitrostyrene. The basic skeleton of DPEN was used as a catalyst, and a catalyst in which one amine was unsubstituted or substituted with 3-pentyl group and the other amine was substituted with thiourea was used.

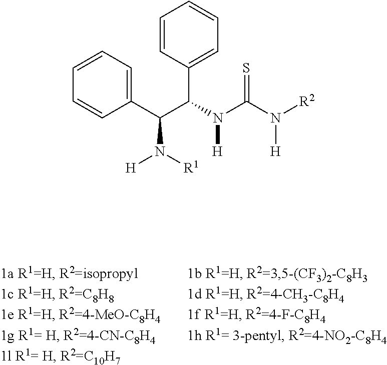

In order to examine the effect of the catalyst, first, a thiourea catalyst in which one amine group was substituted with an alkyl group was used. Toluene was used as the solvent, and the reaction was performed at room temperature (Table 1).

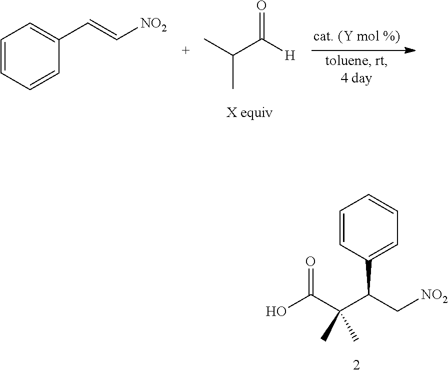

TABLE 1

| | | Temp | | | Yield^{a} | |
|---|---|---|---|---|---|---|
| Entry | Catalyst | (° C.) | Equiv. | mol % | (%) | ee^{b} (%) |
| 1 | 1a | rt | 10 | 10 | 50 | 92 |
| 2 | 1b | rt | 10 | 10 | 91 | 97 |
| 3 | 1c | rt | 10 | 10 | 67 | 93 |
| 4 | 1d | rt | 10 | 10 | 70 | 93 |
| 5 | 1e | rt | 10 | 10 | 50 | 92 |
| 6 | 1f | rt | 10 | 10 | 68 | 97 |
| 7 | 1g | rt | 10 | 10 | 86 | 94 |
| 8 | 1h | rt | 10 | 10 | N.R | — |
| 9 | 1i | rt | 10 | 10 | 45 | 94 |
| 10 | 1b | | 10 | 10 | 74 | 97 |
| 11 | 1b | rt | 7 | 10 | 86 | 97 |
| 12 | 1b | rt | 5 | 10 | 85 | 97 |
| 13 | 1b | rt | 10 | 5 | 83 | 96 |

^{a}isolated yields,
^{b}The ee values were determined by chiral-phase HPLC using OD-H column.

A case of using a thiourea catalyst without an alkyl group in amine was more effective than a case of using a catalyst (Chemical Formula 4-8) in which amine was substituted with an alkyl group. As a result of comparing yields and stereoselectivities of a thiourea catalyst substituted with a paramethoxy group (Chemical Formula 4-5) as an electron donating group (EDG) and a thiourea catalyst substituted with a parafluoro group (Chemical Formula 4-6) as an electron withdrawing group (EWG), the catalyst substituted with the parafluoro group (Chemical Formula 4-6) as the EWG showed higher yield and stereoselectivity. The catalyst substituted with the EWG was higher likely to participate in hydrogen bonds than the catalyst in which hydrogen of thiourea involved in hydrogen bonds was substituted with the EDG, and thus, the stereoselectivity of the product was higher. In particular, the catalyst (Chemical Formula 4-2) substituted with 3,5-bis(trifluoromethyl-1) showed the highest yield and stereoselectivity.

In order to further increase the effect of the catalyst, the reaction was performed at a lower temperature. The reaction at low temperature showed stereoselectivity similar to the reaction at room temperature, but had a lower yield therethan. That is, it was confirmed that the catalyst provided the highest reactivity and stereoselectivity at room temperature.

Meanwhile, the stereoselectivity did not depend on an equivalent of aldehyde and an amount of catalyst, but did affect the reaction yield. As the amount of aldehyde decreased from 10 equiv. to 7 equiv. and 5 equiv., the stereoselectivity was not changed, but the yield was decreased. Subsequently, when 5 mol % of the catalyst was added after fixing 10 equiv. of aldehyde, both the yield and stereoselectivity were decreased compared to the case of using 10 mol % of the catalyst. Accordingly, both yield and optical purity were excellent when 10 equiv. of aldehyde and 10 mol % of the catalyst were used.

### EXPERIMENTAL EXAMPLE 2

### Optimization of Solvent

An experiment was conducted using a catalyst (Chemical Formula 4-2) substituted with 3,5-bis(trifluoromethyl-1), which was the most active in Experimental Example 1. The reaction was performed in all solvents, and a desired yield was obtained in all the solvents except hexane and tetrahydrofuran (THF). The stereoselectivity of 96% or more was obtained in all the solvents. In particular, when water was used as a solvent, the reaction was performed for 12 hours, which was 8 times shorter than other solvents (96 hours), and 5 mol %, which was half the used amount (10 mol %) of catalyst. Therefore, water, which provided the highest yield and stereoselectivity and enabled a Michael addition reaction even at a short reaction time and a small amount of catalyst, was selected as a solvent.

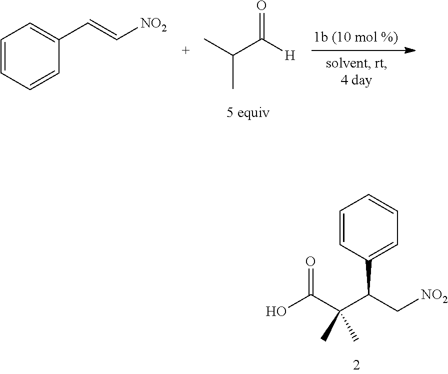

TABLE 2

| Entry | Solvent | Yield (%) ^{a} | ee (%) ^{b} |
|---|---|---|---|
| 1 | n-hexane | 52 | 97 |
| 2 | CHCl₃ | 80 | 97 |
| 3 | THF | 60 | 96 |
| 4 | benzene | 87 | 97 |
| 5 | EtOH | 58 | 97 |
| 6 | toluene | 85 | 97 |
| 7^{c} | water | 99 | 99 |
| 8 | CH₂Cl₂ | 85 | 97 |

^{a} isolated yields,
^{b} The ee values were determined by HPLC using OD-H column.
^{c}The reactions were run with catalyst of 5 mol %, 12 h

### EXPERIMENTAL EXAMPLE 3

### Reaction Depending on Type of carbonyl Compound

3.1. In Case of Ketone

Various ketones and phenyl-substituted nitroalkenes were reacted in the presence of catalysts, solvents, and phenol derivatives (Table 3).

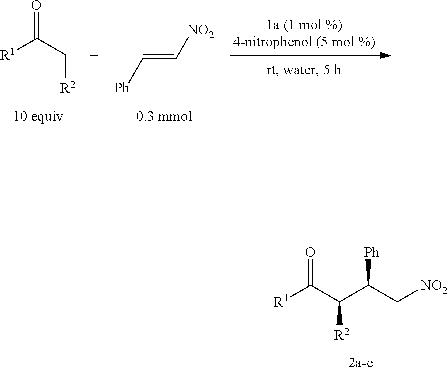

TABLE 3

| | | | | | ee (syn) ^{c} |
|---|---|---|---|---|---|
| Entry | R1 | R2 | Yield ª (%) | syn/anti ^{b} | (%) |
| 1 ^{d} | CH₃ | H | 72 | — | 72 |
| 2 ^{e} | CH₃ | H | 81 | — | 98 |
| 3 ^{f} | CH₃ | H | 90 | — | 99 |
| 4 ^{g} | CH₃ | H | 98 | — | 99 |
| 5 | C₆H₅ | H | 95 | — | 98 |
| 6 | —(CH₂)₄— | 99 | 90/10 | 99 | |
| 7 | —(CH₂)₃— | 98 | 80/20 | 88 | |
| 8 | —CH₂CH₂OCH₂— | 87 | 82/18 | 66 | |
| 9 | —CH₂CH₂SCH₂— | 85 | 66/34 | 94 | |

^{a} isolated yields,
^{b} diastereoselectivities were determined by ¹H NMR analysis,
^{c} The ee values were determined by chiral-phase HPLC,
^{d} used 1 mol % of 1b catalyst,
^{e} used 5 mol % of phenol addictive,
^{f} used 5 mol % of 4-chlorophenol,
^{g} the reaction proceeded for 36 h.

The reaction including acetone (Chemical Formula 1-1) showed relatively high yield and enantioselectivity compared to cycloketone substituted with sulfur (Chemical Formula 1-6) and oxygen (Chemical Formula 1-5). This was because ketone reacted with an amino group of the catalyst to form enamine. In the case of aliphatic ketone, diastereoselectivity and enantioselectivity increased because steric hindrance was small and a nucleophile was easily accessible to nitroalkene.

Meanwhile, when 4-nitrophenol was added as an additive, relatively high yield and enantioselectivity were provided, whereas when phenol and 4-chlorophenol were added, a suitable yield was obtained.

3.2. In Case of aldehyde

Various aldehydes and nitrostyrenes were reacted under optimized conditions (Table 4).

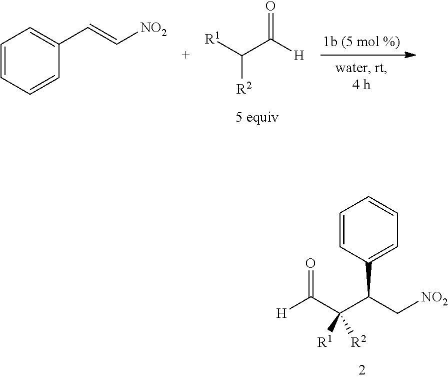

TABLE 4

| | | | | dr ^{b} | |
|---|---|---|---|---|---|
| Entry | R1 | R2 | Yield (%) ª | (syn:anti) | ee (%) ^{c} |
| 1 | Me | H | 95 | 67:33 | 99 |
| 2 | Et | H | 94 | 83:17 | 99 |
| 3 | n-Pr | H | 94 | 83:17 | 98 |
| 4 | i-Pr | H | 93 | 93:07 | 99 |

^{a} isolated yields.
^{b} Determined by ¹H-NMR analysis.
^{c} The ee values were determined by HPLC using OD-H and AD-H columns.

Propionaldehyde (Chemical Formula 1-8) showed high enantioselectivity, butyraldehyde (Chemical Formula 1-9) and pentanal (Chemical Formula 1-10) showed higher diastereoselectivity than propionaldehyde, and 3-methylbutanal (Chemical Formula 1-11) showed the best diastereoselectivity and enantioselectivity. This means that the larger an alkyl group of aldehyde, the higher the diastereoselectivity due to steric hindrance.

### EXPERIMENTAL EXAMPLE 4

### Reaction Depending on Type of nitroalkene

4.1. In Case of aliphatic ketone

To confirm the applicability of the reaction to aliphatic ketone, acetone (Chemical Formula 1-1) reacted with various α,β-unsaturated nitroalkenes (Table 5).

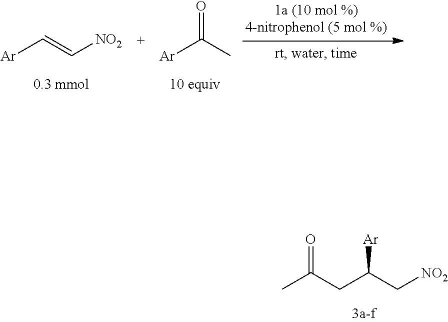

TABLE 5

| Entry | Ar | Time (h) | Yield (%) ª | ee (%) ^{b} |
|---|---|---|---|---|
| 1 | 4-MeC₆H₄ | 10 | 91 | 99 |
| 2 | 4-MeOC₆H₄ | 12 | 95 | 99 |
| 3 | 4-ClC₆H₄ | 9 | 96 | 99 |
| 4 | 4-BrC₆H₄ | 9 | 99 | 98 |
| 5 | 2-MeOC₆H₄ | 17 | 88 | 99 |
| 6 | 2-furyl | 10 | 95 | 98 |

^{a} Isolated yields,
^{b} the ee values were determined by chiral-phase HPLC.

Nitroalkenes (Chemical Formulas 2-4, 2-5, and 2-7) attached with phenyl rings substituted with electron withdrawing groups reacted better overall and had relatively shorter reaction time than nitroalkenes attached with phenyl rings substituted with electron donating groups (entry 3, 4, 6 of Table 2). In the case of the electron withdrawing groups, the reaction time was reduced because the nucleophiles did not prevent approach to electrophiles.

4.2. In Case of cycloketone

The results of reacting cycloketone with various α,β-unsaturated nitroalkenes were shown in Table 6 below.

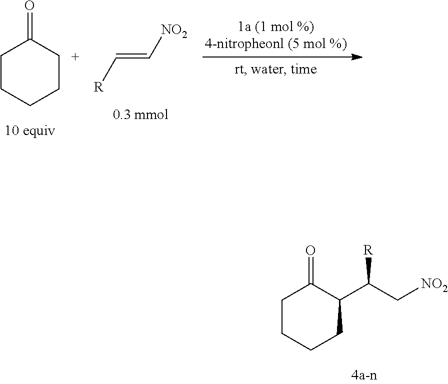

TABLE 6

| Entry | R | Yield ª (%) | syn/anti ^{b} | ee (syn) ^{c} (%) |
|---|---|---|---|---|
| 1 | 4-Me—C₆H₄ | 98 | 86/14 | 99 |
| 2 | 4-i-Pr—C₆H₄ | 88 | 93/7 | 82 |
| 3 | 4-F—C₆H₄ | 92 | 88/12 | 99 |
| 4 | 4-Cl—C₆H₄ | 99 | 81/19 | 99 |
| 5 | 4-Br—C₆H₄ | 97 | 85/15 | 96 |
| 6 | 3-Cl—C₆H₄ | 93 | 91/9 | 96 |
| 7 | 2-furyl | 92 | 92/8 | 96 |
| 8 | 2-thienyl | 99 | 84/16 | 98 |
| 9 | 2-naphthyl | 92 | 89/11 | 92 |
| 10 | 4-MeO—C₆H₄ | 99 | 91/9 | 99 |
| 11 | 2-MeO—C₆H₄ | 94 | 91/9 | 99 |
| 12 | 2-NO2—C₆H₄ | 95 | 87/13 | 94 |

^{a} Isolated yields,
^{b} diastereoselectivities were determined by ¹H NMR analysis,
^{c} the ee values were determined by chiral-phase HPLC

Trans-β-nitrostyrene (Chemical Formula 2-1) had similar or slightly higher enantioselectivity than nitroalkenes (Chemical Formulas 2-4, 2-5, and 2-9) having a halogen-substituted phenyl ring at a para position. Similarly, reactions using nitroalkenes substituted with 2-furyl (Chemical Formula 2-7), 2-thienyl (Chemical Formula 2-11), and 2-naphthyl (Chemical Formula 2-12) had relatively excellent enantioselectivities. In particular, the reaction with the 2-furyl derivative showed the highest yield. In addition, the nitroalkene (Chemical Formula 2-3) attached to a phenyl ring having a methoxy group as an electron donating group at the 4th position showed slightly higher yield and stereoselectivity than a nitro group as an electron withdrawing group at the 4th position.

4.3. In Case of aldehyde

The reactions of isobutyraldehyde (Chemical Formula 1-7) and various α,β-unsaturated nitroalkenes were performed under optimal conditions (Table 7).

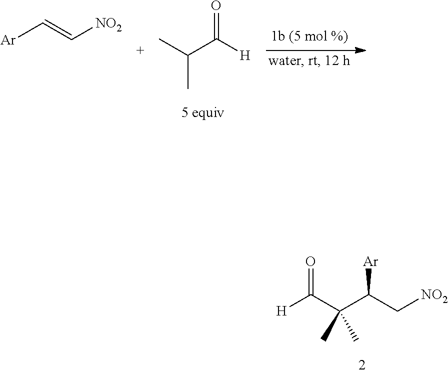

TABLE 7

| Entry | Ar | Yield (%) ^{a} | ee (%) ^{b} |
|---|---|---|---|
| 1 | Ph | 99 | 99 |
| 2 | 4-Cl—Ph | 94 | 99 |
| 3 | 4-Br—Ph | 94 | 98 |
| 4 | 4-Me—Ph | 94 | 99 |
| 5 | 2-Furyl | 96 | 99 |
| 6 | 4-MeO—Ph | 97 | 99 |
| 7 ^{c} | 2-MeO—Ph | 96 | 99 |
| 8 | 4-OH—Ph | 96 | 99 |

^{a} isolated yields,
^{b} The ee values were determined by HPLC using OD-H column.

As a result, the highest yield was obtained when unsubstituted nitrostyrene (Chemical Formula 2-1) was used. In addition, the reaction with nitrostyrenes substituted with an electron withdrawing group and an electron donating group also showed excellent stereoselectivity and yield.

### EXPERIMENTAL EXAMPLE 5

### Calculation of Transition State Energy Through DFT

5.1. Experimental Method

Density functional theory (DFT) calculation showed a mechanism of a substrate and a catalyst and was performed using Gaussian 16 and Gauss-View 6.0 programs. An optimized shape was described using a B3LYP/6-31G(d,p) level. After the shapes of a reactant, an intermediate (IM), a transition state (TS), and a product were completely optimized, zero-point energy (ZPE) was obtained through vibrational frequency calculation at the same theoretical level, and a potential energy surface (PES) was obtained. Enthalpy correction and entropy according to a temperature were calculated at 298 K and 1 atm.

5.2. In Case of ketone

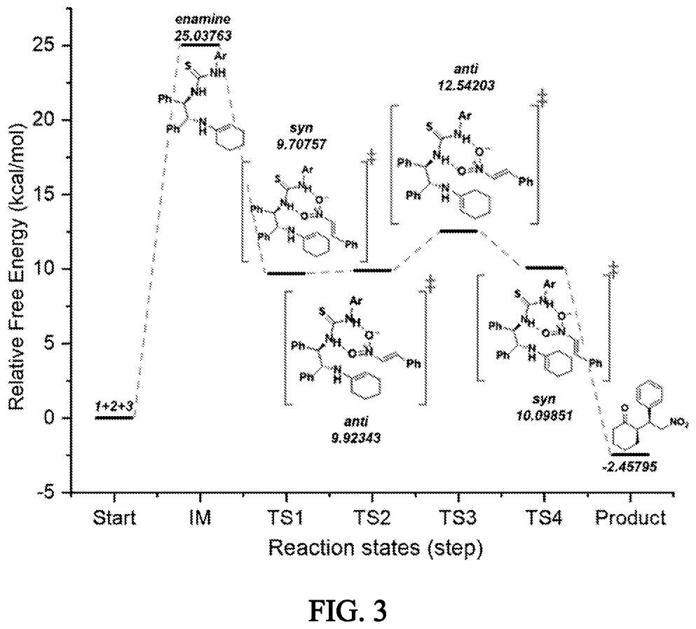

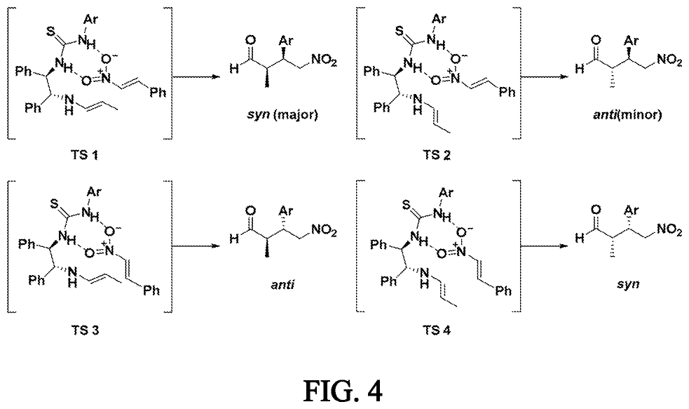

A mechanism proposed for a Michael reaction between cyclohexanone and phenyl-substituted nitroalkene was illustrated in FIG. 2. As a result of calculating the expected energy of transition state 1 (TS 1) through DFT, it was confirmed that a syn structure of TS 1 was the most stable than TS 2 (anti), TS 3 (anti), and TS 4 (syn) based on each Gibbs free energy (FIG. 3). In addition, minor TS 3 showed the highest free energy than other TS structures. In addition, cyclohexanone also reacts with a primary amine of the catalyst to form enamine. TS 1 and TS 3 have little steric hindrance because double bonds and thiourea were located on the same side. The hydrogen bonds between the nitro group of nitroalkene and the thiourea of the catalyst cause aromatic substituents on alkene to be positioned with relatively low steric hindrance as in TS 1 and TS 2. Accordingly, it is expected that nucleophilic enamine approaches an electrophile from the bottom so that a syn(2R, 1S) form is most prevalently produced. In addition, when the ketone and the catalyst react with each other to form enamine, the double bond is formed closer to the nitroalkene, and nitroalkene substituted with an aromatic group is located toward thiourea with relatively little steric hindrance, so that the reaction exhibits relatively high enantioselectivity and diastereoselectivity.

5.3. In Case of aldehyde

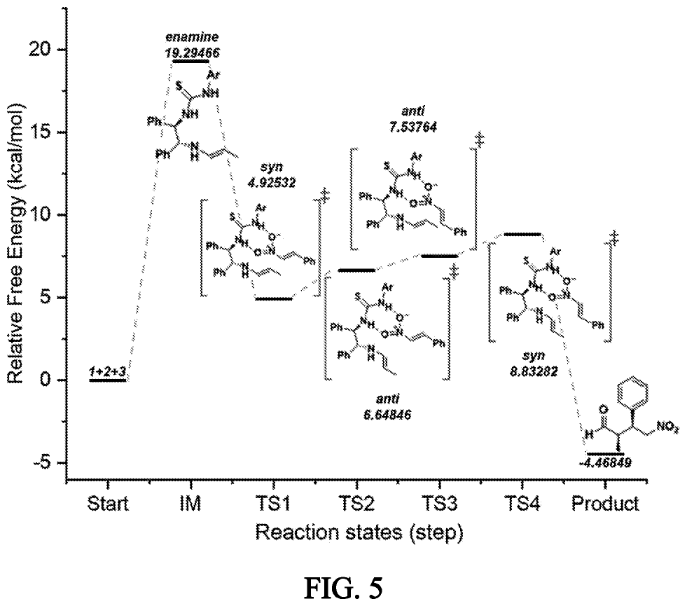

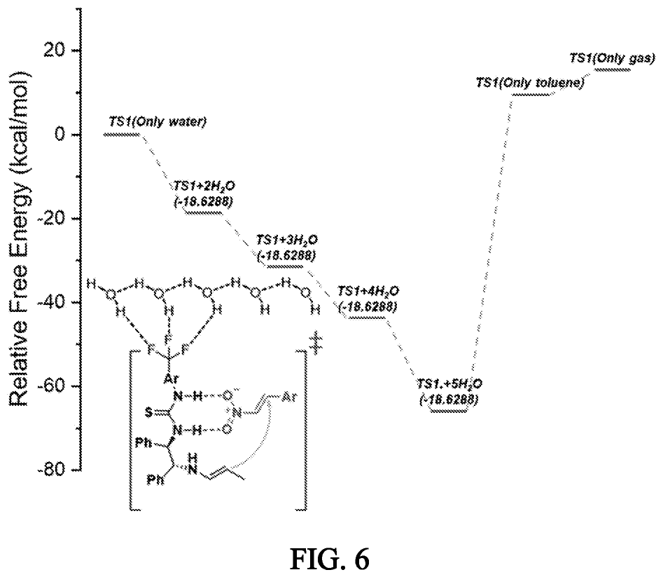

A mechanism proposed for a Michael reaction between propionaldehyde and phenyl-substituted nitroalkene was illustrated in FIG. 4. As a result of calculating the expected energy of transition state 1 (TS 1) through DFT, it was confirmed that a syn structure of TS 1 was the most stable than TS 2 (anti), TS 3 (anti), and TS 4 (syn) based on each Gibbs free energy (FIG. 5). In addition, minor TS 4 showed the highest free energy than other TS structures. In addition, propionaldehyde reacts with the primary amine of the catalyst to form imine and finally form enamine. TS 1 and TS 3 have little steric hindrance because double bonds and thiourea were located on the same side. The hydrogen bonds between the nitro group of nitroalkene and the thiourea of the catalyst cause aromatic substituents on alkene to be positioned with relatively low steric hindrance as in TS 1 and TS 2. Accordingly, it is expected that nucleophilic enamine approaches an electrophile from the bottom so that a syn(2R, 3S) form is most prevalently produced.

5.4. Solvent Effect

The relative freedom of the solvent effect and thermal energy for each step of the Michael reaction were calculated via DFT.

When water as a polar protic solvent was used as a solvent, it was confirmed through experiments that the reactivity was higher than the reaction using other solvents. In addition, when water was used as a solvent, it was confirmed through quantum calculation that the reactivity was improved by stabilizing the transition state of the catalyst. According to the stabilized transition state, aldehyde reacted with an amino group of the catalyst to form enamine, and a thiourea moiety on the other side formed hydrogen bonds with two oxygen atoms of a nitro group. The enamine nucleophile formed by the catalyst attacked the electrophile from below.

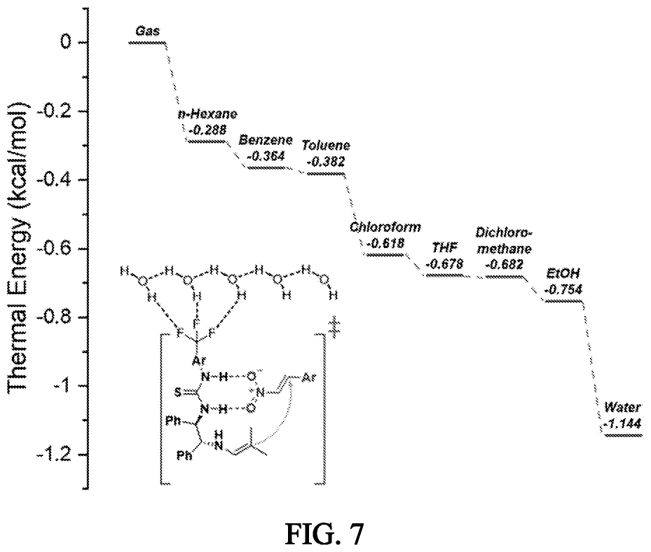

In order to predict a solvent effect of the catalyst, the relative free energy of the transition state (TS) during an interfacial reaction between a hydrophobic substituent (CF₃) of the catalyst 4-2 and H₂O was compared with that of an aqueous binary mixture (H₂O+solvent) and the result thereof was illustrated in FIG. 6.

As a result, it was confirmed that when water was used as the solvent, the relative free energy in the transition state was the lowest. When water was used as the solvent in the Michael addition reaction, the reactivity increased as the polarity of the catalyst increased. This was because the reactivity increased due to the stabilization of the relative energy and the hydrophobic effect of the hydration reaction. In particular, in the case of the catalyst 4-2, the transition state was stabilized through hydrogen bonds between fluorine (F) atoms of the catalyst and hydrogen (H) atoms of water. In addition, when a contact between the catalyst and the water increased due to hydrogen bonds, the degree of stabilization varied according to the number of hydrogen bonds of water molecules (FIG. 6).

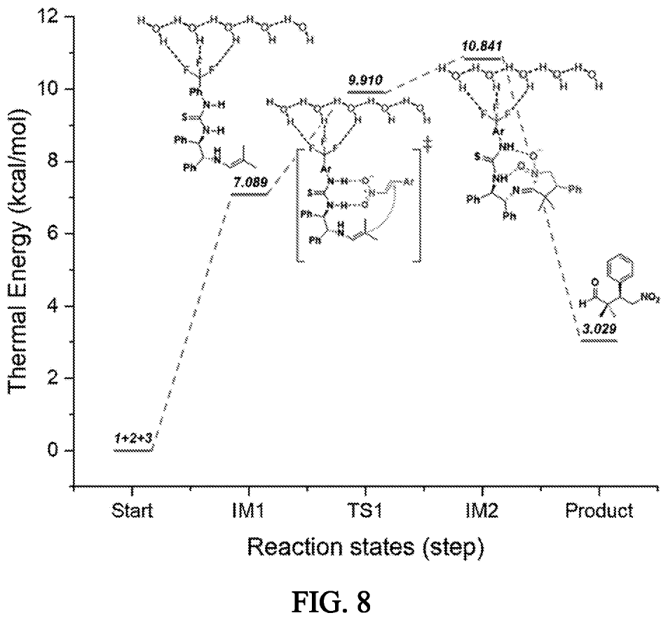

Conventionally, hydrophobic non-polar solvents such as n-hexane and toluene have been widely used in Michael addition reactions. Since the solvent effect on the Michael addition reaction was confirmed in Experimental Example 2 of the present disclosure, thermodynamic analysis was performed to determine a factor affecting the Michael addition reaction of water. Quantum calculations were performed to predict thermophysical data for an interfacial reaction system in the transition state of the catalyst. Comparing an actual reaction (Table 2) and quantum calculation results, it was confirmed that non-polar solvents such as n-hexane and benzene exhibited the lowest reactivity. In addition, CHCl₃, tetrahydrofuran (THF), CH₂Cl₂, and EtOH showed similar reactivity in the calculated results, and it was confirmed that water among these solvents showed the best reaction activity and stability (FIG. 7).

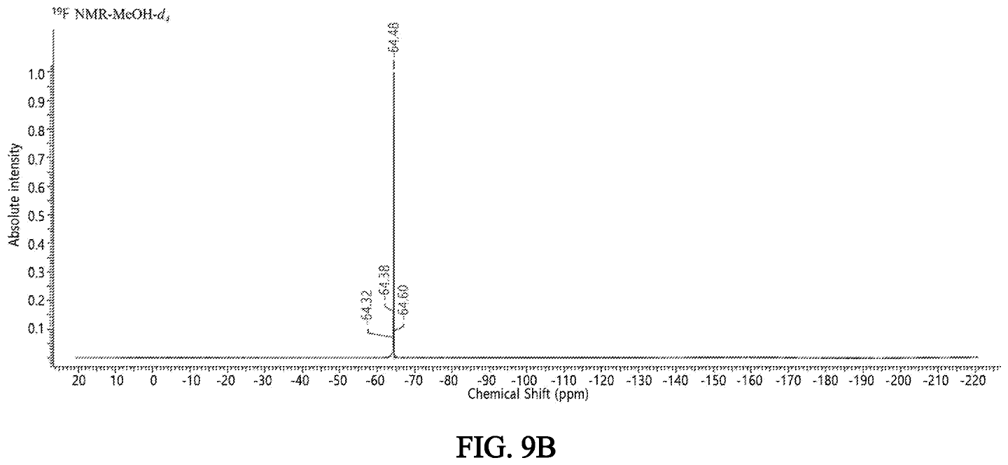

In addition, in order to prove through nuclear magnetic resonance spectroscopy (NMR) that the reaction proceeded through hydrogen bonds between fluorine (F) atoms and hydrogen (H) atoms in a catalytic reaction when water was used as a solvent, as a result of measuring NMR by using DO instead of water, it was confirmed that the peaks for observing fluorine (¹⁹F) in the catalyst shifted depending on the presence or absence of D₂O (FIG. 9A and FIG. 9B). This suggests a possibility of forming hydrogen bonds between fluorine atoms and hydrogen atoms.

As described above, although embodiments have been described by the restricted drawings, various modifications and variations can be applied on the basis of embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result can be achieved.

Therefore, other implementations, other embodiments, and equivalents to the appended claims fall within the scope of the claims to be described below.

What is claimed is:

1. A method for preparing chiral nitro derivatives comprising:
preparing a compound represented by Chemical Formula 3 by reacting a compound represented by Chemical Formula 1 with a compound represented by Chemical Formula 2 in water,
wherein a catalyst compound represented by Chemical Formula 4 is used in the reaction: 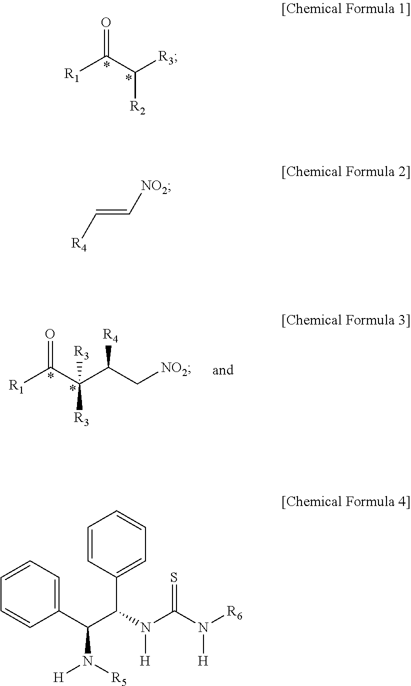
in Chemical Formulas 1 and 3,
R₁ is hydrogen, a C₁-C₇ acyclic alkyl group, or a C₄-C₁₀ aryl group, and
R₂ is hydrogen or a C₁-C₇ acyclic alkyl group, or
the R₁ and R₂ may form a C₄-C₁₀ cycloalkyl group or heterocycloalkyl group together with carbons to which the R₁ and R₂ are attached and carbons marked with asterisks (*), and
R₃ is hydrogen or a C₁-C₇ acyclic alkyl group (however, except when R₁, R₂, and R₃ are all hydrogens),
in Chemical Formulas 2 and 3,
R₄ is a C₄-C₁₀ aryl group or C₄-C₁₀ heteroaryl group unsubstituted or substituted with at least one selected from the group consisting of a halogen group, a hydroxyl group, a nitro group, a C₁-C₇ acyclic alkyl group, a C₁-C₇ alkoxy group, and combinations thereof, and
in Chemical Formula 4,
R₅ is hydrogen or a C₁-C₇ acyclic alkyl group, and
R₆ may be a C₄-C₁₀ aryl group or C₁-C₇ acyclic alkyl group unsubstituted or substituted with at least one selected from the group consisting of a halogen group, a cyano group, a nitro group, a trifluoromethyl group, a C₁-C₇ acyclic alkyl group, a C₁-C₇ alkoxy group, and combinations thereof.

2. The method for preparing the chiral nitro derivatives of claim 1, wherein the reaction is an asymmetric Michael addition reaction.

3. The method for preparing the chiral nitro derivatives of claim 1, wherein the compound represented by Chemical Formula 1 is at least one selected from the group consisting of compounds represented by Chemical Formulas 1-1 to 1-11 below:

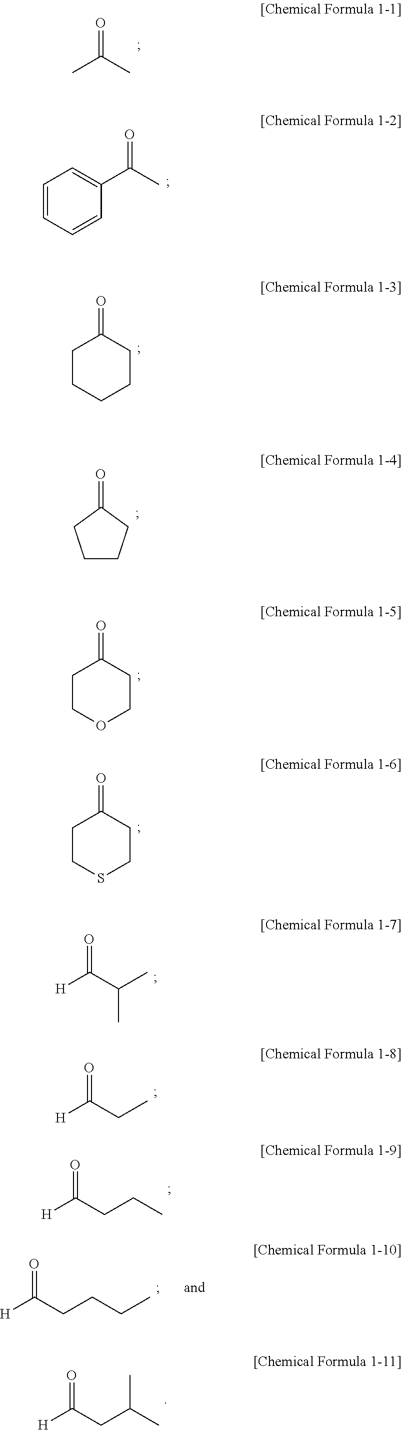

4. The method for preparing the chiral nitro derivatives of claim 1, wherein the compound represented by Chemical Formula 2 is at least one selected from the group consisting of compounds represented by Chemical Formulas 2-1 to 2-14 below:

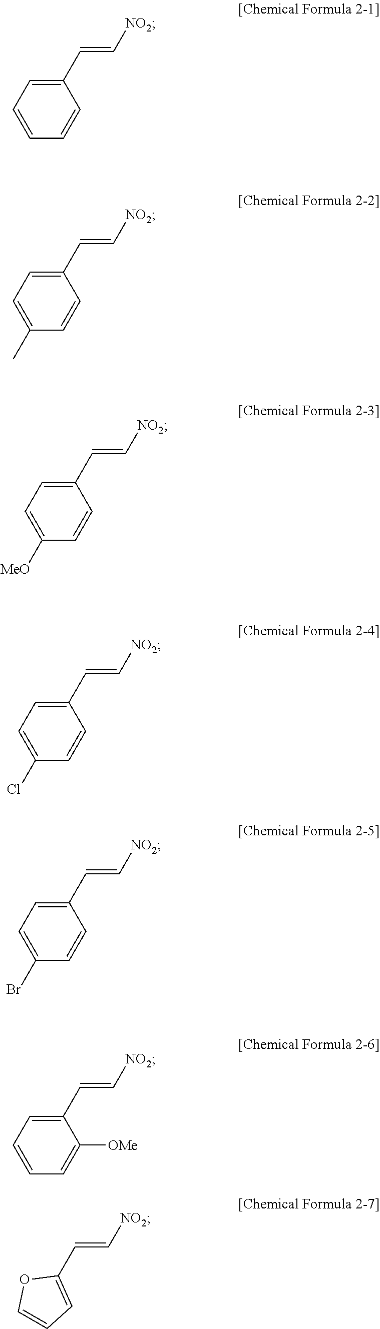

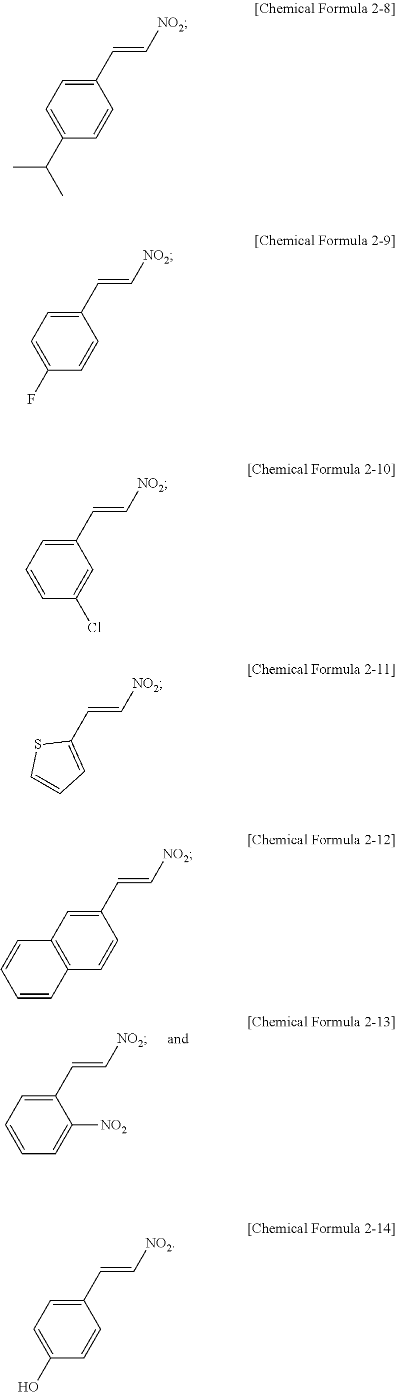

5. The method for preparing the chiral nitro derivatives of claim 1, wherein the compound represented by Chemical Formula 3 is at least one selected from the group consisting of compounds represented by Chemical Formulas 3-1 to 3-36 below:

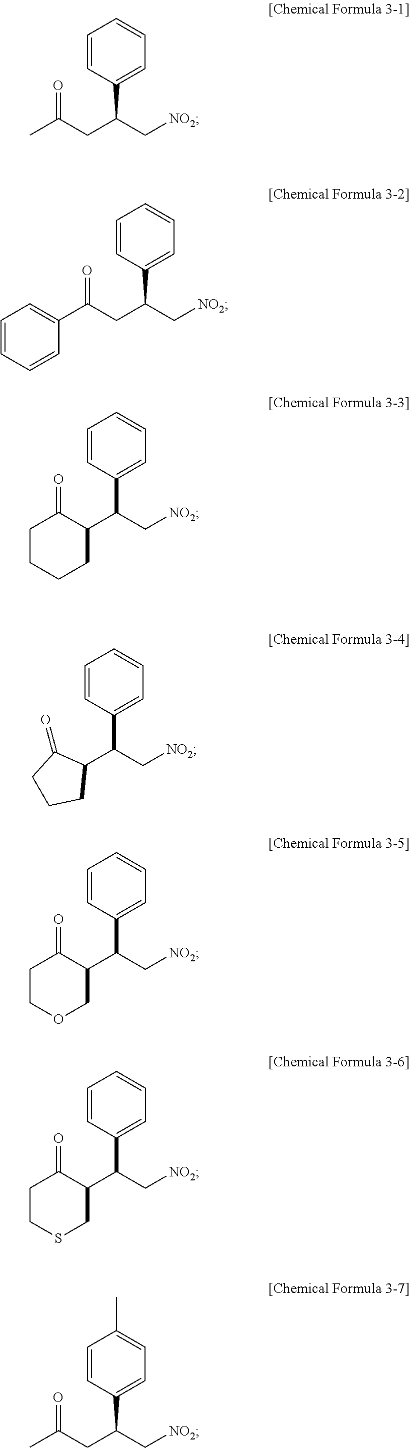

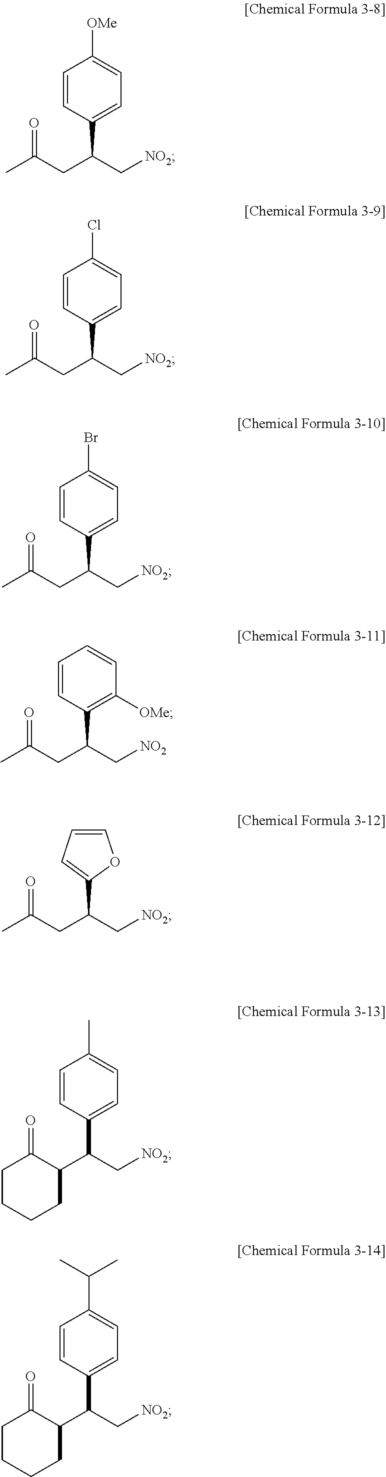

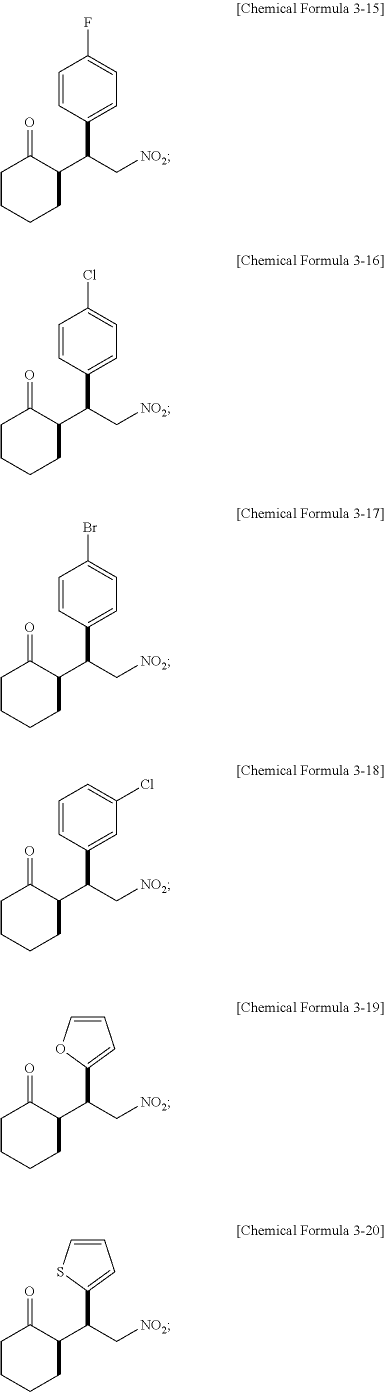

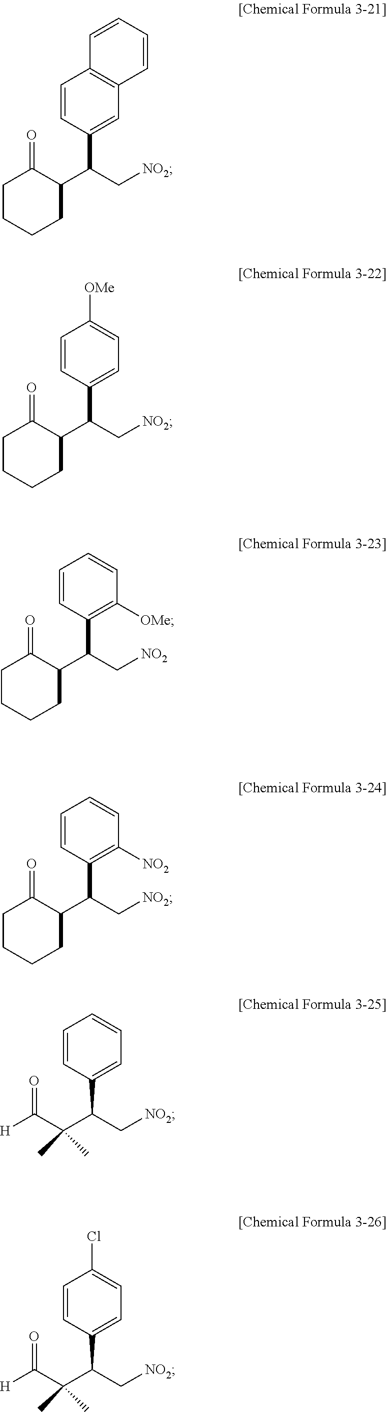

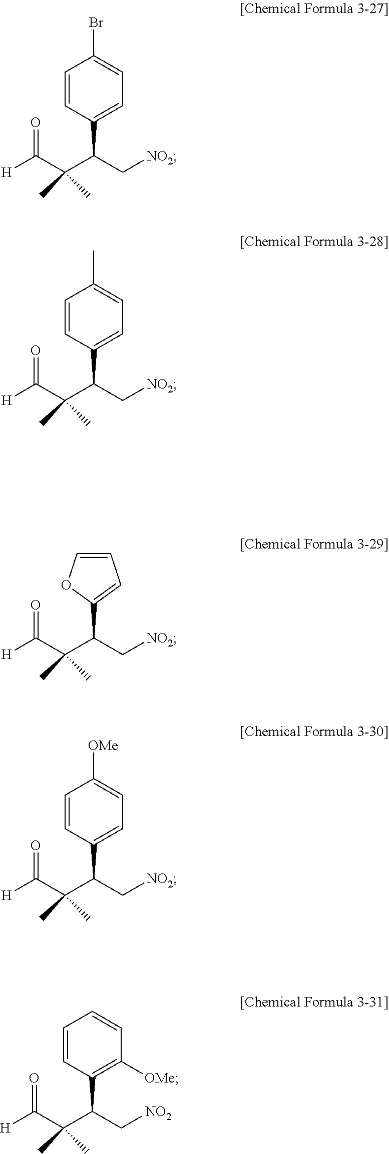

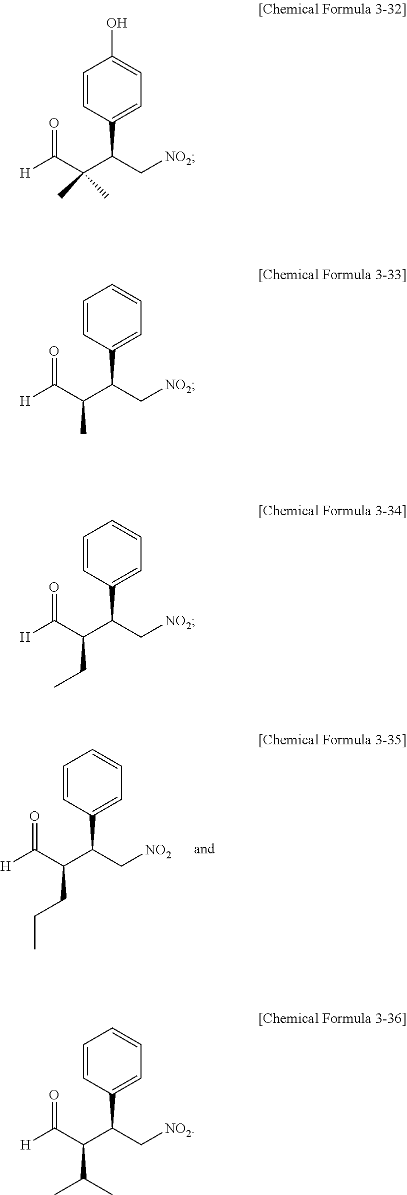

6. The method for preparing the chiral nitro derivatives of claim 1, wherein the compound represented by Chemical Formula 4 is at least one selected from the group consisting of compounds represented by Chemical Formulas 4-1 to 4-9 below:

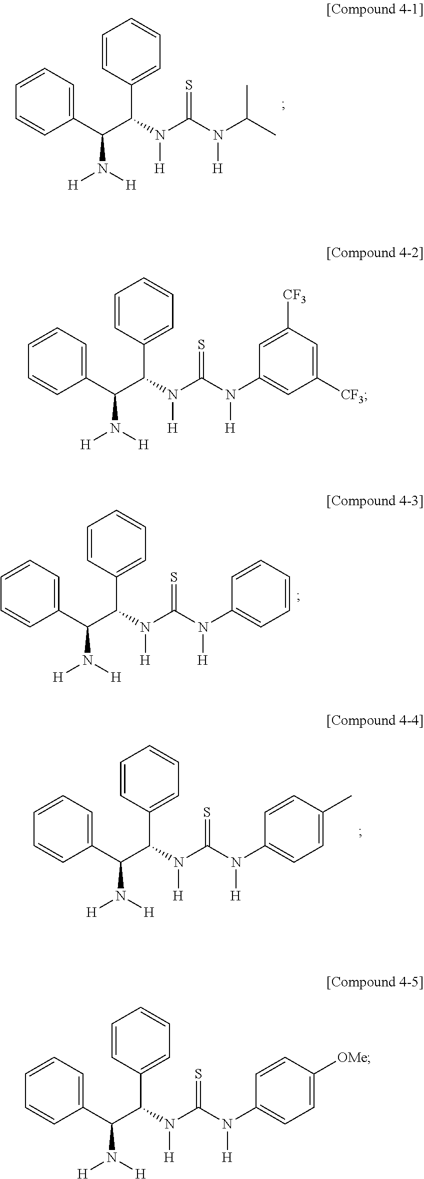 [SMILES: CC(C)NC(=S)N[C@@H](c1ccccc1)[C@@H](N)c1ccccc1 ; N[C@@H](c1ccccc1)[C@@H](NC(=S)Nc1cc(C(F)(F)F)cc(C(F)(F)F)c1)c1ccccc1 ; N[C@@H](c1ccccc1)[C@@H](NC(=S)Nc1ccccc1)c1ccccc1 ; Cc1ccc(NC(=S)N[C@@H](c2ccccc2)[C@@H](N)c2ccccc2)cc1 ; COc1ccc(NC(=S)N[C@@H](c2ccccc2)[C@@H](N)c2ccccc2)cc1]

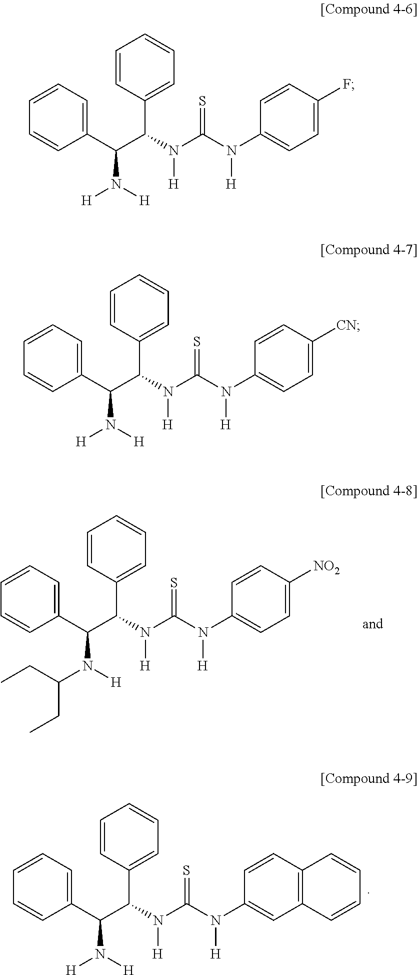 [SMILES: N[C@@H](c1ccccc1)[C@@H](NC(=S)Nc1ccc(F)cc1)c1ccccc1 ; N#Cc1ccc(NC(=S)N[C@@H](c2ccccc2)[C@@H](N)c2ccccc2)cc1 ; CCC(CC)N[C@@H](c1ccccc1)[C@@H](NC(=S)Nc1ccc([N+](=O)[O-])cc1)c1ccccc1 ; N[C@@H](c1ccccc1)[C@@H](NC(=S)Nc1ccc2ccccc2c1)c1ccccc1]

7. The method for preparing the chiral nitro derivatives of claim 1, wherein the compound represented by Chemical Formula 1 and the compound represented by Chemical Formula 2 react with each other by further adding 4-nitrophenol, phenol, or 4-chlorophenol.

8. The method for preparing the chiral nitro derivatives of claim 1, wherein the reaction is performed in one reactor at room temperature.

9. The method for preparing the chiral nitro derivatives of claim 1, wherein 5 to 15 equiv. of the compound represented by Chemical Formula 1 and 0.5 to 2 equiv. of the compound represented by Chemical Formula 2 react with each other.

10. The method for preparing the chiral nitro derivatives of claim 1, wherein 1 to 10 mol % of the catalyst compound represented by Chemical Formula 4 is added.

11. A chiral nitro derivative prepared by the preparation method according to claim 1.

12. A method for preparing chiral indole derivatives comprising:
(1) preparing a compound represented by Chemical Formula 3 by reacting a compound represented by Chemical Formula 1 with a compound represented by Chemical Formula 2 in water using a catalyst compound represented by Chemical Formula 4; and
(2) preparing a compound represented by Chemical Formula 5 from the compound represented by Chemical Formula 3: 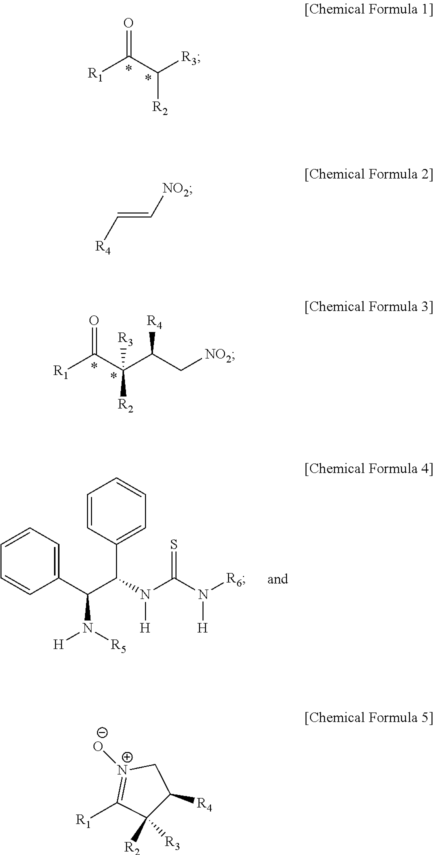
in Chemical Formulas 1, 3, and 5,
R₁ is hydrogen, a C₁-C₇ acyclic alkyl group, or a C₄-C₁₀ aryl group, and
R₂ is hydrogen or a C₁-C₇ acyclic alkyl group, or
the R₁ and R₂ may form a C₄-C₁₀ cycloalkyl group or heterocycloalkyl group together with carbons to which the R₁ and R₂ are attached and carbons marked with asterisks (*), and
R₃ is hydrogen or a C₁-C₇ acyclic alkyl group (however, except when R₁, R₂, and R₃ are all hydrogens),
in Chemical Formulas 2, 3, and 5,
R₄ is a C₄-C₁₀ aryl group or C₄-C₁₀ heteroaryl group unsubstituted or substituted with at least one selected from the group consisting of a halogen group, a hydroxyl group, a nitro group, a C₁-C₇ acyclic alkyl group, a C₁-C₇ alkoxy group, and combinations thereof, and
in Chemical Formula 4,
R₅ is hydrogen or a C₁-C₇ acyclic alkyl group, and
R₆ may be a C₄-C₁₀ aryl group or C₁-C₇ acyclic alkyl group unsubstituted or substituted with at least one selected from the group consisting of a halogen group, a cyano group, a nitro group, a trifluoromethyl group, a C₁-C₇ acyclic alkyl group, a C₁-C₇ alkoxy group, and combinations thereof.

13. The method for preparing the chiral indole derivatives of claim 12, wherein when R₁ and R₂ form a cyclohexyl group together with the carbons to which the R₁ and R₂ are attached and the carbons marked with asterisks (*), the compound represented by Chemical Formula 5 is a compound represented by Chemical Formula 5′:

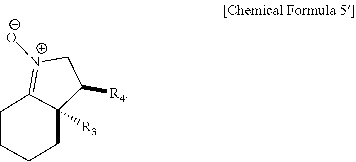

14. The method for preparing the chiral indole derivatives of claim 12, wherein the compound represented by Chemical Formula 3 is added with Zn powder and NH₄Cl.

15. A chiral indole derivative prepared by the preparation method according to claim 12.
